(19) United States
(12) Patent Application Publication
(10) Pub. No.: US 2022/0251604 A1
(43) Pub. Date: Aug. 11, 2022

(54) Oncolytic adenovirus and checkpoint inhibitor combination therapy

(71) Applicant: TILT Biotherapeutics Oy, Helsinki (FI)

(72) Inventors: Akseli Hemminki, Helsinki (FI); Aino Kalervo, Helsinki (FI); Victor Cervera-Carrascon, Helsinki (FI); Riikka Havunen, Helsinki (FI); Joao Viera Lourenco Dos Santos, Helsinki (FI)

(21) Appl. No.: 17/618,932

(22) PCT Filed: Jun. 12, 2020
(86) PCT No.: PCT/FI2020/050422

§ 371 Date: Dec. 14, 2021

Related U.S. Application Data
(60) Provisional application No. 62/861,339, filed on Jun. 14, 2019.
(60) Provisional application No. 62/988,422, filed on Mar. 12, 2020.

(51) Int. Cl. C12N 15/86; A61P 35/00; C07K 16/28

(52) U.S. Cl. CPC C12N 15/86; A61P 35/00; C07K 16/2827; C07K 2317/21; C07K 2317/76; C12N 2710/10332; C07K 16/2878

(57) ABSTRACT

The present invention relates to combination therapy with oncolytic viruses, particularly oncolytic adenoviruses, and checkpoint inhibitors for the treatment of cancer, particularly to a combination comprising (a) an oncolytic adenoviral vector encoding tumor necrosis factor alpha (TNFalpha) and/or interleukin-2 (IL-2) as a transgene and (b) one or more immune checkpoint inhibitors for use in the treatment of cancer.

### FIELD OF THE INVENTION

The present invention relates generally to virology, immunology and medicine. In certain aspects, the invention relates to combination therapy with oncolytic viruses, particularly oncolytic adenoviruses, and checkpoint inhibitors for the treatment of cancer.

### BACKGROUND OF THE INVENTION

Checkpoint inhibitors (CPIs) have revolutionized cancer therapy and validated immunotherapy as an approach. Unfortunately, responses are seen in a minority of patients. Some patients benefit only for a limited time while the majority derive no detectable benefit, especially when it comes to common types of non-melanoma solid tumors. Thus, CPI have definitely been validated as an approach, but since only a minority of patients benefit, there is still an unmet clinical need.

An example of such unmet need is renal cell carcinoma (RCC) for which anti-PD-1 nivolumab has been approved for second line treatment. In a randomized phase 3 trial, the confirmed response rate was 21.5% in the CPI arm versus 3.9% in the everolimus (an inhibitor of the mammalian target of rapamycine) arm. Median overall survival (OS) were 25.0 and 19.1 months, respectively¹. Another clinical trial combined the use of a CPI (atezolizumab, anti-PD-L1) with an anti-VEGF (sunitinib) drug, increasing the overall response ratio (ORR) to 32% (25% for anti-PD-L1 as monotherapy and 29% for sunitinib as monotherapy)². RCC is a tumor type previously described as “immunogenic” and some patients respond to high dose IL-2 treatment³, but the majority show no response to immunotherapies. Clear room for improvement exists in most tumor types, since ORR is still low (melanoma 40%⁴, urothelial carcinoma 21, 1%⁵, non-small cell lung cancer 19.4%⁶, hepatocellular carcinoma 14.3%⁷, among others.

WO2014170389 relates to oncolytic adenoviral vectors alone or together with therapeutic compositions for therapeutic uses and therapeutic methods for cancer. After years of development, the oncolytic viruses are currently starting to be used as cancer therapeutics. Although there have been some discoveries relating to the mechanisms of action and factors that influence the efficacy of the viruses, there is still a need to identify pathways that determine the overall response to virotherapy. In clinical trials, oncolytic viruses have demonstrated a favorable safety profile and promising efficacy. However, there is still room for improvement in the responses, especially in patients with a significant metastasis burden. Further characterization of pathways related to the activity of oncolytic viruses could reveal potential targets for improving the efficacy of virotherapy.

### SUMMARY OF THE INVENTION

The present invention is based on a discovery that co-administration of an oncolytic adenoviral virus coding for cytokines TNFalpha and/or IL-2 and the immune checkpoint inhibitor anti-PD-L1 or PD-1 to clinically relevant cancer models results in a significant shift towards immune activity against the treated cancer concomitant with a high survival benefit relative to administration of either agent alone. Accordingly, in several embodiments, the present application provides a combination therapy for use in the treatment and/or prevention of cancer and/or the establishment of metastases in a mammal and/or for use in initiating, enhancing or prolonging an anti-tumor response in a mammal comprising co-administering to the mammal (a) an oncolytic adenoviral vector encoding TNFalpha and/or IL-2 as a transgene and (b) one or more immune checkpoint inhibitors that preferably binds selectively to PD-L1 or PD-1.

In certain aspects, co-administration of an oncolytic virus and immune checkpoint inhibitor to a subject with cancer provides an enhanced and even synergistic anti-tumor immunity compared to either treatment alone.

In other related aspects, a method for enhancing, potentiating or prolonging the effects of the checkpoint inhibitor or enabling the toxicity or dose or number of treatments of the checkpoint inhibitor to be reduced, comprising administering to a mammal in need thereof (a) an oncolytic adenoviral vector encoding TNFalpha and/or IL-2 as a transgene and (b) one or more immune checkpoint inhibitors that preferably binds selectively to PD-L1 or PD-1, is provided.

### BRIEF DESCRIPTION OF THE DRAWINGS

FIG. 1. Urological cancer patient-derived samples respond to oncolytic virotherapy despite T-cell infiltration and suppression differences. Patient sample 1 is urothelial carcinoma, patient samples 2 and 3 are Clear cell renal cell carcinoma. (A) Hematoxylin and Eosin staining performed on the paraffin-embedded samples. (B) CD8 immunohistochemistry. (C) PD-L1 immunohistochemistry. (D) MTS viability assay after ex vivo treatment of the histocultures. Statistical significances are shown for day 7 calculated by unpaired t-test with Welch's correction (**p<0.01; ***p<0.001). Mean and standard error of the mean (SEM) are shown.

FIG. 2. Responses to virotherapy and checkpoint inhibitor treatments at the cytokine level across 7 days of treatment. Expression values from three individual patient-derived tumor histocultures were plotted together. (A) IFNg. (B) TNFalpha. (C) IL-2. (D) IFNb. (E) Granzyme B. (F) CXCL10. (G) IL-6. (H) TGFb. (I) Arginase. Statistical significances calculated by two-way ANOVA. (*p<0.05; **p<0.01). Mean and standard error of the mean (SEM) are shown.

FIG. 3. Analyses of grouped cytokine responses to virotherapy and checkpoint inhibitor treatments on day 7. Expression values from three different patient-derived tumor histocultures were plotted together. (A) Fold change in immunostimulatory cytokines. (B) Fold change in immunosuppressive effectors. (C) Overall suppression (including IL-6, TGF-b, and arginase) vs overall stimulation (including IFNg, IFNb, Granzyme B, and CXCL10). (D) Pearson's r correlation between immunostimulatory cytokine expression and tumor histoculture viability.

FIG. 4. In vivo testing of virotherapy to enable checkpoint inhibitory therapy. (A) Experimental design: sixty-six animals with B16.OVA tumors started treatments on day 0. Thirty of them were sacrificed on day 7 post-treatment (grey-dashed line) and tumors collected while the rest were kept alive for a survival experiment, with more rounds of treatments until tumors regressed completely or death (S: simultaneous administration of virus and anti-PD-L1, PB: prime and boost). (B) Overall survival (Kaplan-Meier, Log rank Mantel-Cox test). (C-G) Individual tumor growth lines. (*p<0.05; **p<0.01, ***p<0.001).

FIG. 5. A schematic of the adenovirus constructs expressing a single cytokine or two cytokines. The virus backbone is human adenovirus serotype 5, apart from the fiber knob, which is from serotype 3. Both single and double transgenes (TNFalpha, IL-2 or TNFalpha and IL-2) are under transcriptional control of the virus E3 promoter. In case of double transgene, an IRES element separates the two cytokines, resulting in synthesis of each cytokine independently. The transgenes are placed into the E3 region which is deleted for gp19k and 6.7k. The E1A protein is deleted for 24 amino acids (“D24”), in constant region 2, rendering the virus E1A, defective for Rb binding. E1A expression is under regulation of the E2F promoter. E1B/19k bears a disabling deletion.

FIG. 6. Development and validation of an in vivo model refractory to anti-PD-1. (A) Experimental design: 17 mice were engrafted with subcutaneous B16.OVA tumors. When those tumors reached 4 mm in maximum diameter they were assigned to Mock (n=7) or to anti-PD-1 group (n=10). 0.1 mg of anti-PD-1 (or PBS) was given every three days. When tumors progressed over 10 mm, animals were sacrificed. (B) Percentage of animals with a tumor under 10 mm after they started treatment. (C) Individual tumor growth curves for both groups. (Kaplan-Meier, Log rank Mantel-Cox test; ***p<0.001).

FIG. 7. Comparison at the gene expression level of treatment naïve progressing tumors, and tumors progressing after anti-PD-1 therapy. Animals treated as described in FIG. 6 were sacrificed and tumors harvested when those tumors were considered refractory to anti-PD-1. RNA was extracted and expression profiles from both groups were compared. (A) Heatmap and unsupervised clustering of samples. (B) Volcano plot for the expression level comparison between treatment naïve and anti-PD-1 treated tumors. (C) Immune related significantly regulated genes. (Differences in gene regulations were taken into account if fold change was 2 or ≥2, with a q-value 0.001).

FIG. 8. The use of an engineered adenovirus is able to trigger tumor growth control in anti-PD-1 refractory tumors. (A) Experimental design: 29 mice were engrafted with subcutaneous B16.OVA tumors. When those tumors reached 4 mm in maximum diameter, they started receiving 0.1 mg of anti-PD-1 every three days intraperitoneally. When tumors progressed over 8 mm, animals were assigned to a group where animals were treated with the same aPD-1 regimen (n=8), with 1×10⁸ viral particles (vp) intratumorally once every three days (n=8), or both (n=8). Treatments continued until complete responses were observed or sacrifice criteria was reached. (B) Cancer specific survival. (C) Individual tumor growth curves for the groups. (Kaplan-Meier, Log rank Mantel-Cox test; ***p<0.001).

FIG. 9. Generation of tumor samples and analysis to study mechanism of action of the treatments. (A) Experimental design: 27 mice carrying B16.OVA tumors were treated with aPD-1 until they became refractory to the drug as described previously. Subsequently, those animals were assigned to a group where animals were treated with the same aPD-1 regimen (n=9), with 1×10⁸ viral particles (vp) intratumorally once every three days (n=9), or both (n=9). Four rounds of treatments were given at days 0, 1, 3 and 6 after they were considered refractory and sacrificed at day 7 for tumor collection. (B) Average tumor volumes (and SEM) at day 0 (when they qualified as refractory) and day 7 (when tumors were harvested). (C) Heatmap after the analysis of tumors by CyTOF and subsequent processing of the data by FLOWSOM providing 64 different cell clusters for immune (CD45+) cells. (Mann Whitney test; ****p<0.0001).

FIG. 10. Changes in key immune populations after virotherapy assessed after mass cytometry and cluster analysis. Unbiased cell cluster generation from CD45+ fraction, rendered multiple clusters that were associated to a cell type or phenotype. Relative percentage of those clusters among experimental groups were compared using Mann-Whitney test. Key markers to identify the cluster identity are indicated. (A) cluster 25. (B) cluster 41. (C) cluster 10. (D) cluster 17. (E) cluster 6. (F) cluster 14. (G) cluster 36. (H) cluster 5. (I) cluster 39. (J) cluster 58. (K) cluster 32. (L) cluster 55.

FIG. 11. Combination of adenoviruses armed with IL-2 and TNFalpha with anti-PD-L1 improves tumor growth control and survival in a poorly (MOC2) immunogenic murine oral cavity cancer model. (A) Individual tumour volumes normalised to day 0; sample groups: PBS control (PBS), treatment with antiPD-L1 antibody (aPD-L1), treatment with viruses Ad5-CMV-IL2+Ad5-CMV-TNFalpha (Virus), and a combination treatment with antiPD-L1 antibody and viruses Ad5-CMV-IL2+Ad5-CMV-TNFalpha (aPD-L1+ Virus). (B) Mean normalised tumour volume showing improved tumour growth control by day 30. (C) Overall survival curve including median survival for each group. For tumour growth curves statistics were calculated by Mixed Effects Analysis with Tukey's post test (*p<0.05, ***p<0.001). Tumor volume data is presented as mean+SEM (standard error of mean).

FIG. 12. TILT-123 and anti-PD-L1 therapy synergize for rapid tumor cell killing despite histology of patient-derived ovarian cancer samples. MTS viability assay after ex vivo treatment of the ovarian cancer tumor histocultures with 100 viral particles (vp) per cell of Ad5/3-E2F-D24-TNFa-IRES-IL2 (TILT-123), or 20 ug/ml of anti-PD-L1 (aPD-L1), or both, or media (vehicle). OVCA P1 is ovarian low-grade serous carcinoma (Stage IVB), OVCA P2 is ovarian high-grade serous carcinoma (Stage IIIC), OVCA P3 is ovarian clear cell carcinoma (Stage IVB). Statistical significances are shown for day 1 calculated by unpaired t-test with Welch's correction (**p<0.01; ***p<0.001). All data is presented as mean+SEM (standard error of mean).

FIG. 13. TILT-123 induces killing of tumor cells derived from a patient resistant to aPD-1 therapy. MTS viability assay after ex vivo treatment of the ovarian cancer tumor histocultures with 100 viral particles (vp) per cell of Ad5/3-E2F-D24-TNFa-IRES-IL2 (TILT-123), or Ad5/3-E2F-D24, or media (no virus). SCCHN P1 is a brain metastasis from a squamous cell carcinoma of the Head and Neck patient refractory to anti-PD-1 therapy. Statistical significances are shown for day 7 calculated by unpaired t-test with Welch's correction (*p<0.05; **p<0.01). All data is presented as mean+SEM (standard error of mean).

### DETAILED DESCRIPTION OF THE EMBODIMENTS

When an oncolytic adenoviral vector encoding cytokine(s) TNFalpha and/or IL-2 such as TILT-123 (Ad5/3-E2F-d24-hTNFa-IRES-hIL2) and CPI were used together as described in the Experimental Section below, a significant shift towards immune activity (interferon gamma, i.e. IFNg, and granzyme B) and increased T-cell trafficking signals (CXCL10) were observed. As the virus is armed with TNFalpha and IL-2 transgenes, those cytokine levels were also increased in the tumor microenvironment. In vivo, our viruses enabled an anti-PD-L1 (a CPI) to reach 100% complete responses (hazard ratios versus anti-PD-L1 alone 0.057 [0.007; 0.451]) or virotherapy alone 0.067 [0.011; 0.415]), significantly higher than control groups (p<0.01). The same group also showed a significant increase in active CD8 T cells. It has thus been found that combination therapy with an oncolytic adenoviral vector encoding TNFalpha and/or IL-2 as a transgene and a checkpoint inhibitor that selectively binds to PD-L1 results in unexpected improvement in the treatment of cancer. When administered simultaneously or sequentially, the oncolytic virus and the checkpoint inhibitor interact cooperatively and even synergistically to significantly improve survival relative to single administration of either component with no apparent adverse effects or reduction in virus titer. This unexpected effect may allow a reduction in the effective dose of each component, leading to a reduction in side effects and enhancement of clinical effectiveness of the compounds and treatment.

In further experiments (Example 2 below), oncolytic adenoviruses coding for TNFalpha and IL-2 together with an anti-PD-1 antibody were shown to be effective against CPI refractory tumors.

In several embodiments, a combination therapy for use in the treatment of cancer and/or the establishment of metastases in a mammal is provided comprising co-administering to the mammal (i) an oncolytic adenoviral vector encoding TNFalpha and/or IL-2 in combination with (ii) an immune checkpoint inhibitor. In a preferred embodiment, said checkpoint inhibitor selectively binds to PD-L1 or PD-1. In preferred embodiments, said oncolytic adenoviral vector is administered simultaneously or sequentially with the immune checkpoint inhibitor.

Oncolytic Virus

In preferred embodiments, the oncolytic virus of the combination is an oncolytic adenovirus.

As used herein “an oncolytic adenoviral vector” refers to an adenoviral vector capable of infecting and killing cancer cells by selective replication in tumor versus normal cells. WO2014170389 discloses oncolytic adenoviral vectors encoding TNFalpha and/or IL-2 as transgene(s) that can be used in this invention.

The vectors may be modified in any way known in the art, e.g. by deleting, inserting, mutating or modifying any viral areas. The vectors are made tumor specific with regard to replication. For example, the adenoviral vector may comprise modifications in E1, E3 and/or E4 such as insertion of tumor specific promoters (e.g. to drive E1), deletions of areas (e.g. the constant region 2 of E1 as used in “D24”, E3/gp19k, E3/6.7k) and insertion of transgenes. Furthermore, fiber knob areas of the vector can be modified. In one embodiment of the invention the adenoviral vector is Ad5/3 comprising an Ad5 nucleic acid backbone and Ad3 fiber knob or Ad5/3 chimeric fiber knob.

As used herein, expression “adenovirus serotype 5 (Ad5) nucleic acid backbone” refers to the genome of Ad5.

“Ad5/3 vector” refers to a chimeric vector having parts of both Ad5 and Ad3 vectors. In a specific embodiment of the invention, the capsid modification of the vector is Ad5/3 chimerism. As used herein, “Ad5/3 chimeric fiber knob” refers to a chimerism, wherein the knob part of the fiber is from Ad serotype 3, and the rest of the fiber is from Ad serotype 5. Specifically, in one embodiment, the construct has the fiber knob from Ad3 while the remainder of the genome is from Ad5 (SEQ ID NO:5).

One approach for generation of a tumor specific oncolytic adenovirus is engineering a 24 base pair deletion (D24) affecting the constant region 2 (CR2) of E1 (SEQ ID NO:4). In wild type adenovirus CR2 is responsible for binding the cellular Rb tumor suppressor/cell cycle regulator protein for induction of the synthesis (S) phase i.e. DNA synthesis or replication phase. The interaction between Rb and E1A requires eight amino acids (121 to 127) of the E1A protein conserved region, which are deleted in the present invention. The vector of the present invention comprises a deletion of nucleotides corresponding to amino acids 122-129 of the vector according to Heise C. et al. (2000, Nature Med 6, 1134-1139). Viruses with the D24 are known to have a reduced ability to overcome the G1-S checkpoint and replicate efficiently only in cells where this interaction is not necessary, e.g. in tumor cells defective in the Rb-p16 pathway, which includes most if not all human tumors.

It is also possible to replace E1A endogenous viral promoter for example by a tumor specific promoter. In a specific embodiment of the invention hTERT promoter is utilized in the place of DA endogenous viral promoter.

In a specific embodiment, the E1B 19K gene (SEQ ID NO:1), generally known to support replication of adenoviral vectors, has a disabling deletion dE1B 19K (SEQ ID NO:2) in the present vectors. Deletion of E1B 19K is known to sensitize cancer cells to TNFalpha and thus it promotes apoptosis (7).

The sequence for wild-type E1B 19K gene is the following (the deletable region is underlined):

(SEQ ID NO: 1)
atggaggctt gggagtgttt ggaagatttt
tctgctgtgc gtaacttgct ggaacagagc
tctaacagta cctcttggtt ttggaggttt
ctgtggggct catcccaggc aaagttagtc
tgcagaatta aggaggatta caagtgggaa
tttgaagagc ttttgaaatc ctgtggtgag
ctgtttgatt ctttgaatct gggtcaccag
gcgcttttcc aagagaaggt catcaagact
ttggattttt ccacaccggg gcgcgctgcg
gctgctgttg cttttttgag ttttataaag
gataaatgga gcgaagaaac ccatctgagc
ggggggtacc tgctggattt tctggccatg
catctgtgga gagcggttgt gagacacaag
aatcgcctgc tactgttgtc ttccgtccgc
ccggcgataa taccgacgga ggagcagcag
cagcagcagg aggaagccag gcggcggcgg
caggagcaga gcccatggaa cccgagagcc
ggcctggacc ctcgggaatg a

Accordingly, in an embodiment, the sequence for dE1B 19K in the present viral vectors is

(SEQ ID NO: 2)
atggaggctt gggagtgttt ggaagatttt
tctgctgtgc gtaacttgct ggaacagctg
ggtcaccagg cgcttttcca agagaaggtc
atcaagactt tggatttttc cacaccgggg
cgcgctgcgg ctgctgttgc ttttttgagt
tttataaagg ataaatggag cgaagaaacc
catctgagcg gggggtacct gctggatttt
ctggccatgc atctgtggag agcggttgtg
agacacaaga atcgcctgct actgttgtct
tccgtccgcc cggcgataat accgacggag
gagcagcagc agcagcagga ggaagccagg
cggcggcggc aggagcagag cccatggaac
ccgagagccg gcctggaccc tcgggaatga

The E3 region is nonessential for viral replication in vitro, but the E3 proteins have an important role in the regulation of host immune response i.e. in the inhibition of both innate and specific immune responses. The gp19k/6.7K deletion in E3 refers to a deletion of 965 base pairs from the adenoviral E3A region. In a resulting adenoviral construct, both gp19k and 6.7K genes are deleted (Kanerva A et al. 2005, Gene Therapy 12, 87-94). The gp19k gene product is known to bind and sequester major histocompatibility complex I (MHC1, known as HLA1 in humans) molecules in the endoplasmic reticulum, and to prevent the recognition of infected cells by cytotoxic T-lymphocytes. Since many tumors are deficient in HLA1/MHC1, deletion of gp19k increases tumor selectivity of viruses (virus is cleared faster than wild type virus from normal cells but there is no difference in tumor cells). 6.7K proteins are expressed on cellular surfaces and they take part in downregulating TNF-related apoptosis inducing ligand (TRAIL) receptor 2.

Both of these deletions provide an advantage. To regain expression of HLA/MHC for presentation of tumor epitopes, e.g., to the adoptively transferred T cells, expression of the gp19k protein is counterproductive and in fact, the upregulation of HLA/MHC requires deletion of gp19k. With regard to 6.7k, since an embodiment of our invention is production of TNFalpha from the virus, and one of its anti-tumor activities is a direct anti-tumor proapoptotic effect (on both transduced and non-transduced bystander cells), the presence of 6.7k is counterproductive.

In one embodiment of the invention, the cytokine transgene or transgenes are placed into a gp19k/6.7k deleted E3 region, under the E3 promoter. This restricts transgene expression to tumor cells that allow replication of the virus and subsequent activation of the E3 promoter. E3 promoter may be any exogenous (e.g. CMV or E2F promoter, SEQ ID NO:3)) or endogenous promoter known in the art, specifically the endogenous E3 promoter. Although the E3 promoter is chiefly activated by replication, some expression occurs when E1 is expressed. As the selectivity of D24 type viruses occurs post E1 expression (when E1 is unable to bind Rb), these viruses do express E1 also in transduced normal cells. Thus, it is of critical importance to regulate also E1 expression to restrict E3 promoter mediated transgene expression to tumor cells.

In another embodiment of the invention E3 gp19k/6.7k is kept in the vector but one or many other E3 areas have been deleted (e.g. E3 9-kDa, E3 10.2 kDa, E3 15.2 kDa and/or E3 15.3 kDa).

In a specific embodiment of the invention the oncolytic adenoviral vector is based on an adenovirus serotype 5 (Ad5) nucleic acid backbone comprising a 5/3 chimeric fiber knob, and comprising the following: E2F1 promoter for tumor specific expression of E1A, a 24 bp deletion (D24) in the Rb binding constant region 2 of adenoviral E1, a nucleic acid sequence deletion of viral gp19k and 6.7k reading frames, with a transgene insertion into the deleted region, resulting in replication-associated control of transgene expression under the viral E3 promoter, and a nucleic acid sequence encoding at least one cytokine transgene in the place of the deleted adenoviral genes gp19k/6.7K in the E3 region. In one embodiment of the invention, the adenoviral vector is based on a human adenovirus.

The exact functions of the Early Region (E3) proteins in adenovirus 3 are not known. Generally in adenoviruses they do not seem to impair replication when deleted and they seem to affect anti-viral host response to adenoviruses. The E3 of the human adenovirus genome contains the highest level of genetic diversity among the six species (A-F) of adenoviruses found in humans. This diversity in genetic content is primarily located between the highly conserved E3-gp19K and E3-RIDα open reading frames (ORFs) where species-specific arrays of genes are encoded.

Cytotoxic T-cell mediated killing of viral-infected cells is modulated by E3-gp19K. This is accomplished by blocking transport of MHC class I to the plasma membrane, and inhibiting the TAP-MHC class I complex formation.

Thus, in one aspect of the invention the important molecule E3-gp19K is comprised in the adenoviral vector to make virus replication stealthier and enable more time for oncolysis and its beneficial effects. Also, retaining E3-gp19K can reduce induction of anti-adenovirus-cytotoxic T cells, resulting in more anti-tumor T cells.

Cytokines participate in immune response by acting through various mechanisms including recruitment of T cells towards the tumor. The nucleotide sequence encoding a cytokine transgene may be from any animal such as a human, ape, rat, mouse, hamster, dog or cat, but specifically it is encoded by a human sequence. The nucleotide sequence encoding the transgene may be modified in order to improve its effects, or unmodified i.e. of a wild type.

Particular embodiments of the present invention include viral vectors coding for at least one cytokine. In a specific embodiment of the invention the cytokine is IL-2 or TNFalpha, preferably the viral vectors are coding for both cytokines. In one embodiment of the invention the viral vectors are coding for IL-2 and/or TNFalpha and a further cytokine, preferably selected from a group consisting of interferon alpha, interferon beta, interferon gamma, complement C5a, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2.

Cytokine TNFalpha (tumor necrosis factor alpha) functions by attracting and activating the T cells and reducing tumor immunosuppression, while IL-2 (interleukin-2) induces the propagation of the T-cell graft. Thus, IL-2 is produced locally at the tumor where it is needed, instead of injected systemically as is typically done in T-cell therapy, which can cause side effects, and therefore a major problem of the prior art therapies (i.e. toxicity of systemic IL-2) can be prevented by this embodiment.

The danger signaling provided by replication of the oncolytic virus, and activation of pathogen associated molecular pattern recognition receptors by viral DNA, together with the action of the transgene(s) may reduce tumor immunosuppression to such degree that preconditioning therapy can be omitted. Consequently, and major issue in prior art, toxicity due to preconditioning chemotherapy and radiation can be avoided.

In one embodiment of the invention the virus vector comprises an internal ribosomal entry site (IRES) or optionally a ribosome shunt site 2A between the two transgenes. Thus, IRES or a ribosome shunt site 2A may be between any cytokines, such as IL-2 and any other cytokine, preferably selected from the above listed cytokine group. As used herein “IRES” refers to a nucleotide sequence that enables initiation of the translation in the middle of a messenger RNA sequence in protein synthesis. IRES can be from any virus, but in one embodiment of the invention IRES is from encephalomyocarditis virus (EMCV). As used herein “a ribosome shunt site 2A” refers to a translation initiation site in which ribosomes physically bypass parts of the 5′ untranslated region to reach the initiation codon. Both the IRES and the A2 enable viruses to produce two transgenes from one promoter (the E3 promoter).

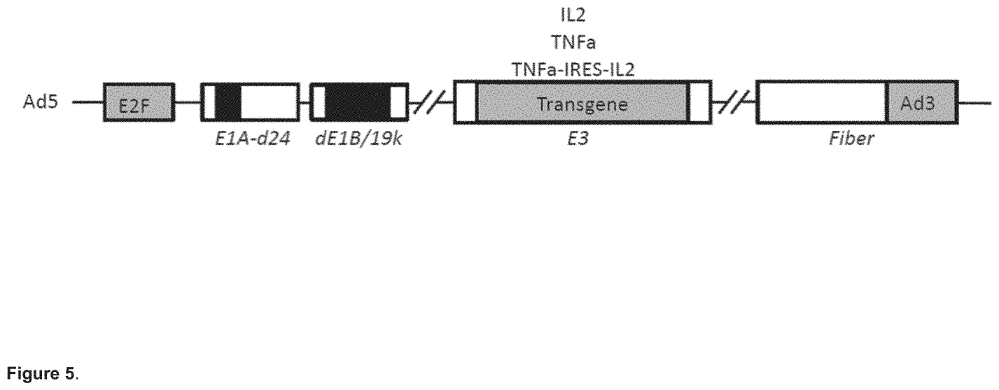

Examples of detailed structures of the oncolytic adenoviral vector encoding TNFalpha and/or IL-2 as a transgene are disclosed in WO2014170389. See also FIG. 5.

In summary, the key advantages of the present invention utilizing viral vectors comprising at least one cytokine transgene are: i) cytokines and virus per se cause a danger signal which recruits T cells and other immune cells to tumors, ii) cytokines induce T-cell proliferation both at the tumor and in local lymphoid organs, iii) cytokines and virus per se are able to induce T cells (both the adoptive T-cell graft and natural, innate anti-tumor T cells) to propagate at the tumor, iv) cytokine and/or virus induce the upregulation of antigen-presenting molecules (HLA) on cancer cells, rendering them sensitive to recognition and killing by T cells, and v) cytokines and virus replication favorably alter tumor microenvironment by reducing immunosuppression and cellular anergy.

The viral vectors utilized in the present inventions may also comprise other modifications than described above. Any additional components or modifications may optionally be used but are not obligatory for the present invention.

Insertion of exogenous elements may enhance effects of vectors in target cells. The use of exogenous tissue or tumor-specific promoters is common in recombinant vectors and they can also be utilized in the present invention.

In summary, the present invention reveals that the replication of oncolytic virus can recruit T cells and induce danger signals at the tumor, reducing immunosuppression and cellular anergy. These effects are mediated through pathogen associated molecular pattern recognition receptors, an evolutionarily conserved mechanism for inducing immunity and not subject to tolerance. The present invention also reveals that an added benefit of the oncolytic platform, capable of replication in tumors but not normal cells, is self-amplification at the tumor. In addition, the oncolytic effect per se may add to the overall anti-tumor effect in humans.

Checkpoint Inhibitor

Immune checkpoint proteins interact with specific ligands which send a signal into T cells that inhibits T-cell function. Cancer cells exploit this by driving high level expression of checkpoint proteins on their surface thereby suppressing the anti-cancer immune response.

A checkpoint inhibitor (also referred to as a CPI) as described herein is any compound capable of inhibiting the function of an immune checkpoint protein. Inhibition includes reduction of function as well as full blockade. In particular, the immune checkpoint protein is a human checkpoint protein. Thus, the immune checkpoint inhibitor is preferably an inhibitor of a human immune checkpoint.

Checkpoint proteins include, without limitation, CTLA-4, PD-1 (and its ligands PD-L1 and PD-L2), B7-H3, B7-H4, HVEM, TIM3, GAL9, LAG3, VISTA, KIR, BTLA, TIGIT and/or IDO. The pathways involving LAG3, BTLA, B7-H3, B7-H4, TIM3 and KIR are recognized in the art to constitute immune checkpoint pathways similar to the CTLA-4 and PD-1 dependent pathways. The immune checkpoint inhibitor can be an inhibitor of CTLA-4, PD-1 (and its ligands PD-L1 and PD-L2), B7-H3, B7-H4, HVEM, TIM3, GAL9, LAG3, VISTA, KIR, BTLA, TIGIT and/or IDO. In some embodiments, the immune checkpoint inhibitor is an inhibitor of PD-L1 or PD-1.

In some embodiments, the checkpoint inhibitor of the combination is an antibody. The term “antibody” as used herein encompasses naturally occurring and engineered antibodies as well as full length antibodies or functional fragments or analogs thereof that are capable of binding e.g. the target immune checkpoint or epitope (e.g. retaining the antigen-binding portion). The antibody for use according to the methods described herein may be from any origin including, without limitation, human, humanized, animal or chimeric and may be of any isotype with a preference for an IgG1 or IgG4 isotype and further may be glycosylated or non-glycosylated. The term antibody also includes bispecific or multispecific antibodies so long as the antibody(s) exhibit the binding specificity herein described. Preferably, the immune checkpoint inhibitor is a monoclonal antibody that selectively binds to PD-L1, more preferably selected from the group consisting of: BMS-936559, LY3300054, atezolizumab, durvalumab and avelumab. Examples of monoclonal antibodies that bind to human PD-1, are described in U.S. Pat. Nos. 7,521,051, 8,008,449, and 8,354,509. Specific anti-human PD-1 mAbs useful as the PD-1 antagonist in the treatment method include: pembrolizumab (MK-3475), nivolumab (BMS-936558), and the humanized antibodies h409A11, h409A16 and h409A17, which are described in WO2008156712.

Humanized antibodies refer to non-human (e.g. murine, rat, etc.) antibodies whose protein sequences have been modified to increase similarity to a human antibody. Chimeric antibodies refer to antibodies comprising one or more element(s) of one species and one or more element(s) of another specifies, for example a non-human antibody comprising at least a portion of a constant region (Fc) of a human immunoglobulin.

Many forms of antibody can be engineered for use in the combination of the invention, representative examples of which include an Fab fragment (monovalent fragment consisting of the VL, VH, CL and CHI domains), an F(ab′)2 fragment (bivalent fragment comprising two Fab fragments linked by at least one disulfide bridge at the hinge region), a Fd fragment (consisting of the VH and CHI domains), a Fv fragment (consisting of the VL and VH domains of a single arm of an antibody), a dAb fragment (consisting of a single variable domain fragment (VH or VL domain), a single chain Fv (scFv) comprising the two domains of a Fv fragment, VL and VH, that are fused together, eventually with a linker to make a single protein chain.

In some embodiments, checkpoint inhibitors (also referred to as CPIs) of the combination therapy are antibodies or fragments thereof that specifically bind to an immune checkpoint protein PD-L1 or PD-1. In particularly preferred embodiments, the immune checkpoint inhibitor is a monoclonal antibody, a fully human antibody, a chimeric antibody, a humanized antibody or fragment thereof that capable of at least partly antagonizing PD-L1 or PD-1.

Cancer

The recombinant vectors of the present invention are replication competent in tumor cells. In one embodiment of the invention the vectors are replication competent in cells, which have defects in the Rb-pathway, specifically Rb-p16 pathway. These defective cells include all tumor cells in animals and humans. As used herein “defects in the Rb-pathway” refers to mutations and/or epigenetic changes in any genes or proteins of the pathway. Due to these defects, tumor cells overexpress E2F and thus, binding of Rb by E1A CR2, that is normally needed for effective replication, is unnecessary. Further selectivity is mediated by the E2F promoter, which only activates in the presence of free E2F, as seen in Rb/p16 pathway defective cells. In the absence of free E2F, no transcription of E1A occurs and the virus does not replicate. Inclusion of the E2F promoter is important to prevent expression of E1A in normal tissues, which can cause toxicity both directly and indirectly through allowing transgene expression from the E3 promoter.

The present invention relates to approaches for treating cancer in a subject. In one embodiment of the invention, the subject is a human or a mammal, specifically a mammal or human patient, more specifically a human or a mammal suffering from cancer.

The approach can be used to treat any cancers or tumors, including both malignant and benign tumors, both primary tumors and metastases may be targets of the approach. In one embodiment of the invention the cancer features tumor-infiltrating lymphocytes. The tools of the present invention are particularly appealing for treatment of metastatic solid tumors featuring tumor-infiltrating lymphocytes. In another embodiment the T-cell graft has been modified by a tumor or tissue specific T-cell receptor of chimeric antigen receptor.

As used herein, the term “treatment” or “treating” refers to administration of at least oncolytic adenoviral vectors and checkpoint inhibitors that selectively binds to PD-L1 to a subject, preferably a mammal or human subject, for purposes which include not only complete cure but also prophylaxis, amelioration, or alleviation of disorders or symptoms related to a cancer or tumor. Therapeutic effect may be assessed by monitoring the symptoms of a patient, tumor markers in blood, or for example a size of a tumor or the length of survival of the patient

In another embodiment of the invention the cancer or tumor is selected from a group consisting of nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head and neck cancer, eye cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer. Preferably, the cancer or tumor treated is selected from the group consisting of renal cancer, ovarian cancer, bladder cancer, prostate cancer, breast cancer, colorectal cancer, lung cancer (such as small-cell lung carcinoma, non-small-cell lung carcinoma and squamous non-small-cell lung carcinoma), gastric cancer, classical Hodgkin lymphoma, mesothelioma, and liver cancer. In a more preferred embodiment, the cancer or tumor type is head and neck cancer, most preferably human head and neck cancer.

Before classifying a human or animal patient as suitable for the therapy of the present invention, the clinician may examine a patient. Based on the results deviating from the normal and revealing a tumor or cancer, the clinician may suggest treatment of the present invention for a patient.

In an embodiment of the invention, the subject or patient has already failed at least one previous chemotherapy or immunotherapy treatment such as a CPI treatment, i.e. the cancer of the patient is a checkpoint inhibitor (CPI) refractory tumor. In a preferred embodiment, the present invention is directed to the treatment of a CPI refractory tumor. Without wishing to be bound by a theory, the present experimental results show that the genes mediating CD8+ T-cell activity and thus relating to immune activity such as genes for T-cell precursors GZMG and GMZF, genes for T-cell proteins relevant in the interaction with other cell types like KLRC2, genes for immune components such as complement CD46, and particularly genes for T-cell activity regulators such as TNFSF18/GITRL and EAR2, are downregulated in CPI refractory cancer cells in relation to cells naïve to the CPI therapy and thus this change in gene expression may reflect the refractory phenotype. Accordingly, in an embodiment, the present invention is directed to a treatment of CPI refractory cancers capable of mediating or causing dysfunctionality and/or inactivity of CD8+ T-cells.

Pharmaceutical Composition

A pharmaceutical composition of the invention comprises at least one type of viral vectors of the invention. Preferably, the present invention provides a pharmaceutical composition containing (a) an oncolytic virus in combination with (b) a checkpoint inhibitor. The present invention also provides said pharmaceutical combination for use in the treatment of cancer. Furthermore, the composition may comprise at least two, three or four different vectors. In addition to the vector and checkpoint inhibitor, a pharmaceutical composition may also comprise other therapeutically effective agents, any other agents such as pharmaceutically acceptable carriers, buffers, excipients, adjuvants, additives, preservatives, antiseptics, filling, stabilising and/or thickening agents, and/or any components normally found in corresponding products. Selection of suitable ingredients and appropriate manufacturing methods for formulating the compositions belongs to general knowledge of a man skilled in the art.

The pharmaceutical composition may be in any form, such as solid, semisolid or liquid form, suitable for administration. A formulation can be selected from a group consisting of, but not limited to, solutions, emulsions, suspensions, tablets, pellets and capsules. The compositions of the current invention are not limited to a certain formulation, instead the composition can be formulated into any known pharmaceutically acceptable formulation. The pharmaceutical compositions may be produced by any conventional processes known in the art.

A pharmaceutical kit of the present invention comprises an oncolytic adenoviral vector encoding TNFalpha and/or IL-2 as a transgene and one or more immune checkpoint inhibitors that selectively binds to PD-L1 or PD-1. The oncolytic adenoviral vector encoding TNFalpha and/or IL-2 as a transgene is formulated in a first formulation and said one or more immune checkpoint inhibitors that selectively binds to PD-L1 or PD-1 are formulated in a second formulation. In another embodiment of the invention the first and the second formulations are for simultaneous or sequential, in any order, administration to a subject. In another embodiment, said kit is for use in the treatment of cancer or tumor.

Administration

The vector or pharmaceutical composition of the invention may be administered to any mammal subject. In a specific embodiment of the invention, the subject is a human. A mammal may be selected from a group consisting of pets, domestic animals and production animals.

Any conventional method may be used for administration of the vector or composition to a subject. The route of administration depends on the formulation or form of the composition, the disease, location of tumors, the patient, comorbidities and other factors. Accordingly, the dose amount and dosing frequency of each therapeutic agent in the combination depends in part on the particular therapeutic agent, the severity of the cancer being treated, and patient characteristics. Preferably, a dosage regimen maximizes the amount of each therapeutic agent delivered to the patient consistent with an acceptable level of side effects. In a preferred embodiment, the checkpoint inhibitor is administered in an amount from about 2 mg/kg to 50 mg/kg, more preferably about 2 mg/kg to 25 mg/kg.

In one embodiment of the invention, the separate administration(s) of (a) an oncolytic adenoviral vector encoding TNFalpha and/or IL-2 as a transgene and (b) one or more immune checkpoint inhibitors that preferably binds selectively to PD-L1 or PD-1 to a subject is (are) conducted simultaneously or consecutively, in any order. This means that (a) and (b) may be provided in a single unit dosage form for being taken together or as separate entities (e.g. in separate containers) to be administered simultaneously or with a certain time difference. This time difference may be between 1 hour and 1 week, preferably between 12 hours and 3 days, more preferably up to 24 or 48 hours. In a preferred embodiment, the first administration of the adenoviral vector is conducted before the first administration of the checkpoint inhibitor. In addition, it is possible to administer the virus via another administration way than the checkpoint inhibitor. In this regard, it may be advantageous to administer either the virus or checkpoint inhibitor intratumorally and the other systemically or orally. In a particular preferred embodiment, the virus is administered intratumorally and the checkpoint inhibitor intravenously. Preferably, the virus and the checkpoint inhibitor are administered as separate compounds. Concomitant treatment with the two agents is also possible.

As used herein “separate administration” or “separate” refers to a situation, wherein (a) an oncolytic adenoviral vector encoding TNFalpha and/or IL-2 as a transgene and (b) one or more immune checkpoint inhibitors that preferably binds selectively to PD-L1 or PD-1 are two different products or compositions distinct from each other.

Only one combined administration of (a) an oncolytic adenoviral vector encoding TNFalpha and/or IL-2 as a transgene and (b) one or more immune checkpoint inhibitors that preferably binds selectively to PD-L1 or PD-1 may have therapeutic effects. There may be any period between the administrations depending for example on the patient and type, degree or location of cancer. In one embodiment of the invention there is a time period of one minute to four weeks, specifically 1 to 10 days, more specifically 1 to five days, most specifically up to 24 or 48 hours between the consecutive administration of (a) an oncolytic adenoviral vector encoding TNFalpha and/or IL-2 as a transgene and (b) one or more immune checkpoint inhibitors that preferably binds selectively to PD-L1 or PD-1 and/or there are several administrations of a) an oncolytic adenoviral vector encoding TNFalpha and/or IL-2 as a transgene and (b) one or more immune checkpoint inhibitors that preferably binds selectively to PD-L1 or PD-1. The numbers of administration times of (a) an oncolytic adenoviral vector encoding TNFalpha and/or IL-2 as a transgene and (b) one or more immune checkpoint inhibitors that preferably binds selectively to PD-L1 or PD-1 may also be different during the treatment period. Oncolytic adenoviral vectors or checkpoint inhibitors may be administered for example from 1 to 10 times in the first 2 weeks, 4 weeks, monthly or during the treatment period. In one embodiment of the invention, administration of vectors or any compositions is done three to seven times in the first 2 weeks, then at 4 weeks and then monthly. In a specific embodiment of the invention, administration is done four times in the first 2 weeks, then at 4 weeks and then monthly. In another specific embodiment, administration of the adenoviral vector is carried out three times (in one embodiment the first dose is given intravenously, second and third dose intratumorally) and of the checkpoint inhibitor one time or two times during the first four weeks, then both the viral vector and the checkpoint inhibitor are administered once per month. The length of the treatment period may vary, and for example may last from two to 12 months or more.

In a specific embodiment of the invention (a) an oncolytic adenoviral vector encoding TNFalpha and/or IL-2 as a transgene and (b) one or more immune checkpoint inhibitors that preferably binds selectively to PD-L1 or PD-1 are administered on the same day and thereafter oncolytic adenoviral vectors are administered every week, two weeks, three weeks or every month during a treatment period which may last for example from one to 6 or 12 months or more.

In one embodiment of the invention, the administration of oncolytic virus is conducted through an intratumoral, intra-arterial, intravenous, intrapleural, intravesicular, intracavitary or peritoneal injection, or an oral administration. Any combination of administrations is also possible. The approach can give systemic efficacy despite local injection. Checkpoint inhibitors may be administered intravenously or intratumorally. In one embodiment the administration of the checkpoint inhibitors is conducted through an intratumoral, intra-arterial, intravenous, intrapleural, intravesicular, intracavitary or peritoneal injection, or an oral administration.

The effective dose of vectors depends on at least the subject in need of the treatment, tumor type and location of the tumor and stage of the tumor. The dose may vary for example from about 1×10⁸ viral particles (VP) to about 1×10¹⁴ VP, specifically from about 5×10⁹ VP to about 1×10¹³ VP and more specifically from about 3×10⁹ VP to about 2×10¹² VP. In one embodiment oncolytic adenoviral vectors coding for at least one cytokine are administered in an amount of 1×10¹⁰-1×10¹⁴ virus particles. In another embodiment of the invention the dose is in the range of about 5×10¹⁰-5×10¹¹ VP.

Any other treatment or combination of treatments may be used in addition to the therapies of the present invention. In a specific embodiment the method or use of the invention further comprises administration of concurrent or sequential radiotherapy, chemotherapy, antiangiogenic agents or targeted therapies, such as alkylating agents, nucleoside analogs, cytoskeleton modifiers, cytostatic agents, monoclonal antibodies, kinase inhibitors or other anti-cancer drugs or interventions (including surgery) to a subject.

The terms “treat” or “increase”, as well as words stemming therefrom, as used herein, do not necessarily imply 100% or complete treatment or increase. Rather, there are varying degrees of which one of ordinary skill in the art recognizes as having a potential benefit or therapeutic effect.

### Other Embodiments

The present invention is also directed to (a) an oncolytic adenoviral vector encoding TNFalpha and/or IL-2 as a transgene and (b) one or more immune checkpoint inhibitors for use in the treatment of cancer or tumor. Preferably, a human cancer or tumor.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

It will be obvious to a person skilled in the art that, as the technology advances, the inventive concept can be implemented in various ways. The invention and its embodiments are not limited to the examples described below but may vary within the scope of the claims.

TABLE 1
The sequences listed in the appended Sequence listing.

| SEQ ID NO: | Name: |
|---|---|
| 1 | E1B 19K |
| 2 | dE1B 19K |
| 3 | E2F |
| 4 | D24 |
| 5 | 5/3 knob modification |

### EXPERIMENTAL SECTION

### Example 1

Materials and Methods

Human Tumor Histocultures from Urological Tumor Samples

Urological samples were collected from surgically removed kidneys and turned into single-cell suspension following previously described methodology(9). Single-cell cultures were treated with 100 viral particles (vp) of Ad5/3-E2F-d24-hTNFa-IRES-hIL2 per cell, 20 μg/mL of anti-human PD-L1 (Atezolizumab, Roche), or the both in triplicates. Cytokine production and cell viability were assessed after 1, 3 and 7 days.

Histopathology Analyses

Hematoxylin and eosin (H&E), and CD8 (clone 4611, CD8-4611-L-CE-H, Novocastra) stainings were performed on patient samples and analyzed by a trained pathologist. For PD-L1 expression assessment, the PD-L1 VENTANA (SP142) assay (Roche) was performed by a trained pathologist. Histopathological analyses from murine samples were carried out by a veterinary pathologist as described previously(10).

Cell Viability Assay

Human tumor histocultures were treated (as described before) up to 7 days. Cell viability was assessed with Cell Titer 96 AQueous One Solution Cell Proliferation Assay (Promega, G3582), following manufacturer indications. The viability of mock-treated cells was set to 100%.

In Vivo Experiments

To study treatment-induced changes in tumors, 2.5×10⁵ B16.OVA melanoma cells were implanted subcutaneously on 4-6 week old female C57BL/6JOIaHsd mice (Envigo Labs). Eleven days after engraftment, animals were randomized into groups (n=12-14/group). Then they received systemic treatments of 0.1 mg of anti-PD-L1 (clone 10F.9G2, 6E0101, BioXCell) and intratumoral injections of 1×10⁸ vp (including equal amounts of Ad5-CMV-mIL2 and Ad5-CMV-mTNFa viruses, non-replicative in mice) on days 0, 1, 3, and 6. PBS was injected intratumorally for groups that did not receive virus. On day 7, 6 animals per group were sacrificed and tumors collected to investigate immune cell phenotyping and cytokines signatures.

The rest of the animals (n=6-8/group) continued to a 90-day OS study, where the treatments continued once every three days, until maximum tumor size reached (18 mm) or complete tumor regression.

Cell Lines and Viruses

B16.OVA, a mouse melanoma cell line was cultured under recommended conditions (8). The cytokine-armed murine adenoviruses' (Ad5-CMV-mIL2 and Ad5-CMV-mTNFa) construction and production has been described previously (12) and were used in the in vivo experiments. For the human tumor histoculture experiments, the oncolytic Ad5/3-E2F-d24-hTNFa-IRES-hIL2 (known as TILT-123) (11) was used.

Cytokine Analyses

Cell culture medium supernatants coming from tumor histocultures were collected after 1, 3, and 7 days. Sample size for the cytokine analyses was restricted on availability of tumor sample (mock; n=4, aPD-L1; n=6, virus; n=3, virus+aPD-L1 (S); n=2, virus+aPD-L1 (PB); n=0). The cytokine levels (IFNg, TNFa, IL-2, IFNb, Granzyme B, CXCL10, IL-6, Arginase, and TGF-b1) on the samples were assessed with a custom Legendplex panel (Biolegend) and a Free Active/Total TGF-b1 detection kit (740488, 740486 and 740487, Biolegend). Cytometric Bead Array Mouse Th1/Th2/Th17 Cytokine kit (560485, BD) was used to study murine tumor samples as described before (8). Both cytokine bead arrays were analyzed with Accuri® (BD). The obtained cytokine values were normalized to total protein concentration of the sample.

PD-L1/2 Expression Assays

When studying PD-L1 expression dynamics, B16.OVA cells were treated with 1000 U/mL of murine IFNg (315-05, Peprotech), a known inducer for PD-L1 expression. Mock control cells were left untreated. After 24 hours of culture, a fraction of the cells were checked for PD-L1 expression and the rest were washed twice with PBS and passed to duplicate 12-well plates Part of the cells previously treated with IFNg stopped receiving the treatment (“withdrawn” group) and the other part continued with the treatment (“IFNg kept” group). The plates were analyzed 24 h and 72 h after the plating.

Similarly, the effect of IFNg (1000 U/mL), TNFa (1000 U/mL and 10000 U/mL), IL-2 (1000 U/mL and 10000 U/mL), Ad5-luc (1 and 100 vp/cell), Ad5-CMV-mIL2 (1 and 100 vp/cell), Ad5-CMV-mTNFa (1 and 100 vp/cell) or different combinations of those on PD-L1 and PD-L2 expression on B16.OVA cells. These expression levels were studied at 24 and 72-hour time points.

Flow Cytometry

Cell cultures and tumor samples were processed and labelled as described elsewhere (8). Anti-CD4-FITC (clone GK1.5, 100406, Biolegend), anti-CD3e-PE (clone 145-2C11, 12-0031-82, eBioscience), anti-CD69-PE-Dazzle (clone H1.2F3, 104536, Biolegend), anti-CD8-PE-Cy5 (clone 53.6-7, 100710, Biolegend), anti-PD-1-PE-Cy-7 (clone 29F.1A12, 135216, Biolegend), anti-CD45-FITC (clone 30-F11, 103107, Biolegend), anti-PD-L2-PE (clone MIH5, 558091, BD), anti-Gr-1-PE-Dazzle (RB6-8C5, 108452, Biolegend), anti-CD11b-PE-Cy5 (M1/70, 101210, Biolegend), anti-PD-L1-PE-Cy7 (TY-25, 107214, Biolegend) were used for flow cytometric analyses under manufacturer's indications. The analyses were performed with s SH800Z cytometer (SONY).

Statistical Analyses

Tumor growth evolution was studied by mixed-model analysis of log-transformed tumor volumes with SPSS Statistics 25 (IBM). GraphPad software was used to present the data, to analyze OS (Kaplan-Meier, Log rank Mantel-Cox test), Hazard Ratios (HRs), and 95% confidence intervals (CIs). In addition, GraphPad was used to evaluate the differences between groups in cytometry or cytokine analyses (unpaired t-test with Welch's correction), correlation analyses between variables (Pearson's r), over-time evolution of variables (two-way ANOVA), and linear regression. Synergy was calculated using the fractional tumor volume (FTV) method. P values <0.05 were considered statistically significant.

Results

Oncolytic Adenovirus Mediated Tumor Cell Lysis in Patient Derived Urological Tumor Histocultures

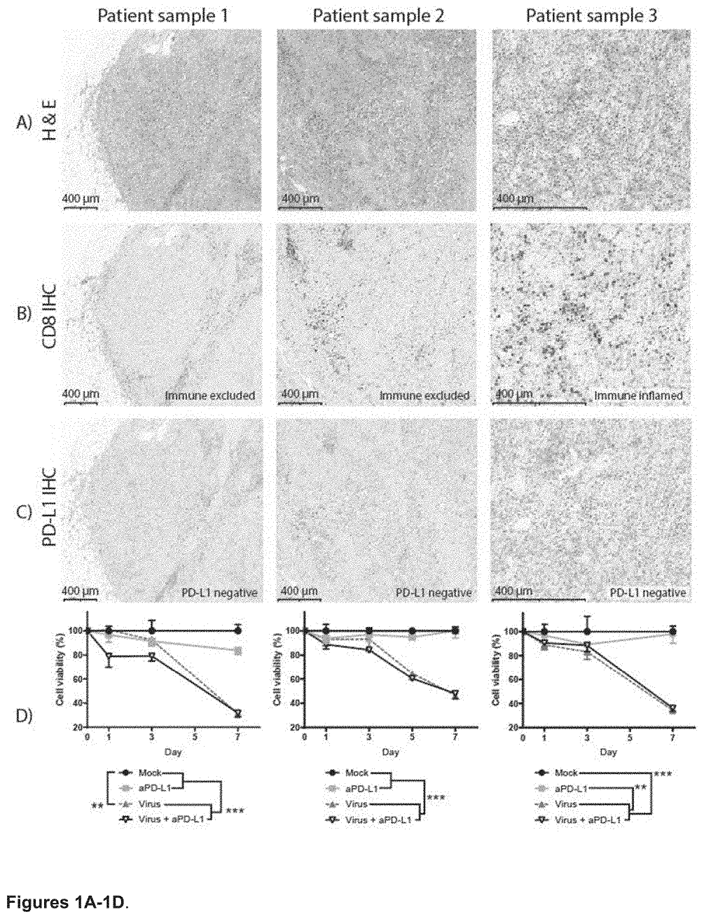

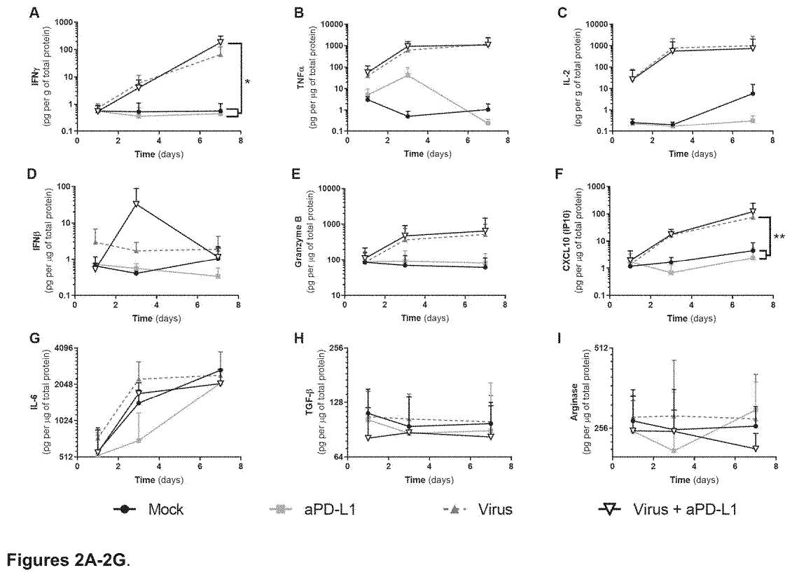

To understand how oncolytic virotherapy can enable responses to checkpoint inhibitors in solid tumors, surgically removed tissues were studied after pathological analysis. These tumors were pathological grade 3 (patient sample 2) or 4 (patient samples 1 and 3) as evaluated from the hematoxylin and eosin staining (FIG. 1A). Because one of the primary effects of the virally delivered cargo affects CD8+ T cells, their presence was also assessed (FIG. 1B), showing two samples with an immune-excluded (“cold”) phenotype (patient samples 1 and 2) and one with an immune-inflamed (“hot”) phenotype (patient sample 3). In addition, of relevance for anti-PD-L1 targeting, an assay to determine PD-L1 positivity was performed. This showed PD-L1 expression below 5% in immune cells in all samples, making them negative according to the test guidelines (FIG. 1C).

After sample processing, a viability assay was performed to measure how the treatments affected the survival of tumor histocultures (FIG. 1D). By day 7, a statistically significant decrease in tumor viability was achieved with virotherapy in all three samples when compared with mock or anti-PD-L1 monotherapy (p<0.01). At that time, the viability of the virally treated groups dropped 62% (95% CI=[54.19; 69.89]) compared with mock and 56% (95% CI=[38.49; 73.39]) when compared with anti-PD-L1 treated samples. Already on day 1 there were significant reductions on cell viability in two of the samples, when virus and anti-PD-L1 were given together.

Oncolytic Virotherapy Triggers a Broad Immunostimulatory Response in Human Urological Tumor Histocultures

We also studied the histocultures for the impact of the treatments on cytokine levels (FIG. 2). Because of the heterogeneity typically seen in actual patient tumors, cytokine levels between samples varied. Nevertheless, groups receiving virotherapy displayed a clear trend for increased expression of immunostimulatory cytokines (IFNg, TNFalpha, IL-2, Granzyme B and CXCL10) (FIGS. 2A-F). Regarding IFNg, increased production was achieved when Ad5/3-E2F-d24-hTNFa-IRES-hIL2 was administered together with anti-PD-L1 in comparison with “Mock” (p=0.0182) and “anti-PD-L1” alone groups (p=0.0181). The “Virus” alone group had a similar trend (p=0.068). TNFa and IL-2 production was also increased in virally treated groups and the differences were significant when samples were analyzed individually. IFNb production, on the other hand, was not as clearly affected as the other immunostimulatory cytokines, except for a peak in production at day 3 with the double treatment.

Granzyme B and CXCL10 had higher expression values in virally treated groups. The expression of these two cytokines correlated with IFNg expression (IFNg/Granzyme B: r=0.629 [0.376; 0.792], p<0.001. IFNg/CXCL10: r=0.494 [0.198; 0.708], p=0.002). CXCL10 expression was significantly increased in “Virus” group when compared with “Mock” (p=0.003) and with “anti-PD-L1” (p=0.002). Treatment with aPD-L1 alone did not affect the expression of immunostimulatory cytokines compared with “Mock” group.

Regarding immunosuppressive mediators (IL-6, TGF-b, and arginase), effects of treatments are not as clear as for the stimulatory ones (FIG. 2A-F). However, statistically significant reduction of IL-6 was achieved by “anti-PD-L1” group and “Virus+anti-PD-L1” when compared with “Mock” or “Virus” in two out of three samples. For TGF-b significant decreases were achieved in all samples for the “Virus+anti-PD-L1” group, while for “anti-PD-L1” group this was seen only in one sample.

Arginase was included in the panel, despite the fact not being a cytokine but an enzyme affecting immune activity. For this enzyme, one sample displayed reduced expression in the “anti-PD-L1” group and “Virus+anti-PD-L1” when compared with “Mock” or “Virus”.

Treatment-Induced Immunostimulatory Cytokine Production Relates with Reduction of Viability in Solid Tumor Samples

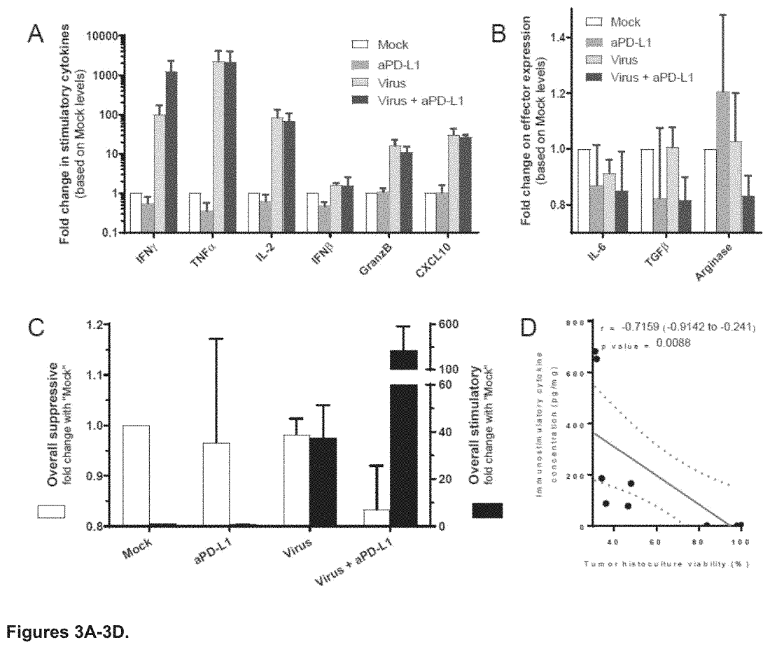

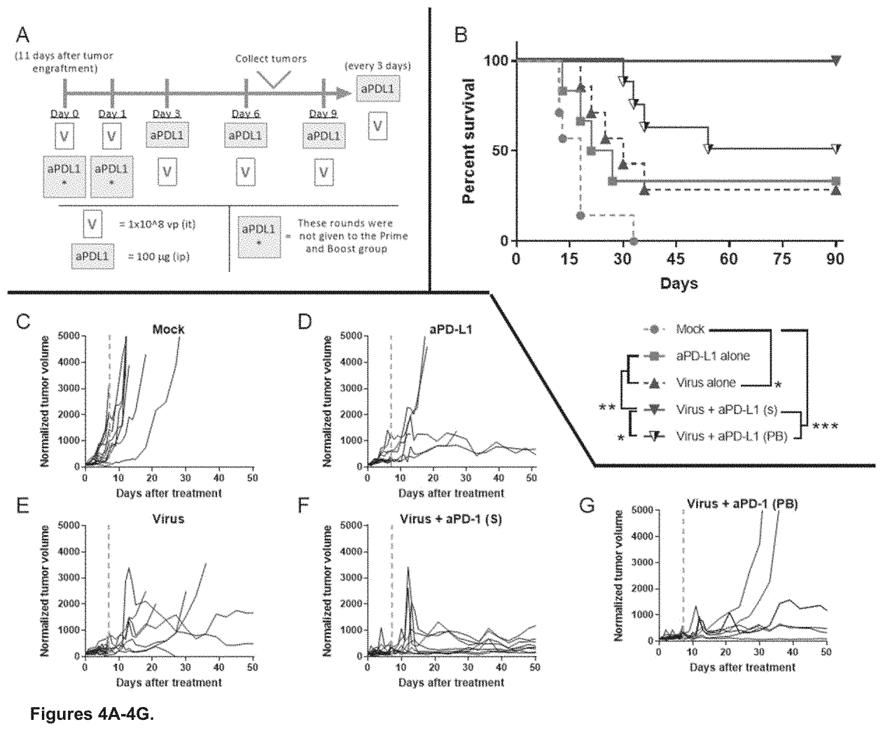

To compare differential expression of cytokines and arginase in tumor histocultures on day 7, average fold change compared with “Mock” were plotted side-by-side (FIGS. 3A and B). Among the six stimulatory cytokines analyzed, the increase in TNFalpha and IL-2 expression is likely related, at least partially, to viral expression of the transgenes and cannot be distinguished from the endogenous production. Of note, there was more than a 1000-fold increase in TNFalpha expression and around a 100-fold increase for IL-2. Regarding IFNg, virally treated groups displayed over a 100-fold increase compared with mock conditions. Interestingly, the addition of anti-PD-L1 to virus treatment results in 10 times higher expression when compared to virus alone (1000 times higher than in “Mock”). Additionally, virus treatments induced an average of 25 fold increase in CXCL10 levels.

The treatments had a less drastic effect on the expression levels of IL-6, TGF-b, and arginase (FIG. 3B). Anti-PD-L1 therapy seemed to reduce the amount of IL-6 and TGF-b, while arginase expression was reduced only when both the virus and the checkpoint inhibitor were given together. To compare the effect of the treatments on endogenously produced immune-suppressors and immune-stimulators, average values of IL-6, TGF-b, and arginase were plotted against average values of IFNg, IFNb, Granzyme B, and CXCL10 (FIG. 3C). The use of both treatments together induced a decrease in immune suppression, delivered by the checkpoint inhibitor, and immunostimulation via virotherapy. An inversely proportional correlation was seen between the production of immunostimulatory cytokines and the tumor histoculture cell viability (r=−0.716 [−0.914; −0.241], p=0.009). No correlation was found between cell viability and immunosuppressive cytokines.

TNFalpha and IL-2 Expressing Adenoviruses Enable Anti-PD-L1 Therapy In Vivo Rendering 100% Complete Response Ratio

Next, we aimed to assess if immune-stimulation triggered by the virus and reduced suppression achieved by anti-PD-L1 was reproducible in vivo. In addition, we wanted to study the impact of tumor immune remodeling in terms of anti-tumor efficacy (FIG. 4A). To understand the interaction between the treatments, two groups were treated with viruses and checkpoint inhibitors but with different administration regimens; while one group received both treatments simultaneously (S) the other received the checkpoint inhibitors only after two rounds of virus treatment, in a “prime and boost” manner (PB).

The two groups that received viruses and anti-PD-L1 together displayed better results in terms of survival (FIG. 4B). Particularly, when the viruses (intratumoral) were administered simultaneously with anti-PD-L1 (intraperitoneal), 100% complete response rate was achieved. The “Virus+aPD-L1 (s)” group had significantly longer survival than any other group (p<0.001 against “Mock, p=0.007 against “aPD-L1”, p=0.005 against “Virus” and p=0.025 against “Virus+aPD-L1 (PB)). The hazard ratio of the “Virus+aPD-L1 (s)” strategy was superior over any other group studied (HR=0.033 [0.006; 0.181] against “Mock, HR=0.057 [0.007; 0.451] against “aPD-L1”, HR=0.067 [0.011; 0.415] against “Virus” and HR=0.104 [0.014; 0.752] against “Virus+aPD-L1 (PB)).

The “prime and boost” approach resulted in significantly longer overall survival (OS) (p<0.001) and lower hazard ratio (HR) (0.059 [0.012; 0.290]) over mock. Virotherapy or checkpoint inhibitor monotherapy resulted in circa 33% complete responses. OS with virotherapy alone was statistically improved over mock (p=0.016), with a lower HR (0.180 [0.044; 0.727]). Individual tumor volume graphs were also plotted (FIG. 4C) and synergistic effects were seen following dual therapy as early as day 5. At day 90 after the treatments started, some of the animals that had undergone a complete response had a scar tissue in the peritumoral area. After sacrifice of the animals, scars were collected and analyzed by a pathologist, who reported the presence of melanophages, plasma cells and lymphocytes, but no malignant cells.

### DISCUSSION

In this study, we show how a viral platform coding for TNFalpha and IL-2 has considerable impact on the immune tumor microenvironment, which led to higher responses in the context of anti-PD-L1 checkpoint inhibition. Similar results were observed both in human urological clinical sample histocultures (renal cell cancer and urothelial cancer) and in vivo, resulting in improved tumor growth control and survival. It is noteworthy that both monotherapies had a positive effect. When they were used together, synergy was clear in the context of tumor control.

In patients, complete responses are quite rare, partial responses are more common, but even taken together, ORR with CPIs are between 10-40% following monotherapy in most solid tumor types. However, it has been noted that TIL presence (1, 2) and upregulated inflammatory cytokine signature (3-5) are among the strongest predictive factors (6). In this regard, it is potentially important the studied viral platform was able to make “cold” tumors “hot”, to enable effective CPI therapy, for an increased response rate and survival.

### Example 2

Materials and Methods

In Vivo Experiments

In vivo experiments were carried out in C57BL/6OIaHsd female mice 4-6 week old at the moment of initiation (purchased from Envigo Labs, Huntingdon, UK). 2.5×10⁵ B16.OVA melanoma cell lines were implanted subcutaneously in the left lower flank and then checked for presence of palpable tumors with at least 4 mm in the longer diameter. When the minimum tumor size criterion was met, animals were randomized into different treatment groups. Tumor volumes were measured daily and overall health was assessed. Animals having open wounds (i.e. ulcers at the injection site) were immediately euthanized. Maximum allowed tumor volume was 18 mm, after which animals were immediately euthanized. Animals with no observable tumors were kept alive at least 90 days after they received the first treatment to ensure no tumor recurrence.

Antibodies and Viruses

Treatment diagrams are provided for each specific experiment. Anti-PD-1 antibody (aPD-1) treatments consisted of systemically (intraperitoneal) delivered antibody dosed as 0.1 mg (clone 10F.9G2, BE0101, BioXCell, Lebanon, N.H., USA) diluted in PBS. Virotherapy treatments consisted of 1×10⁸ viral particles (including equal amounts of Ad5-CMV-mIL2 and Ad5-CMV-mTNFa viruses, non-replicative in mice).

Transcriptome Analyses

Tumors harvested from in vivo experiments were stabilized in RNAlater (R0901, Sigma-Aldrich, St. Louis, Mo., USA) and stored at −20° C. Following RNeasy (74104, Qiagen, Hilden, Germany) kit manufacturer's guide, RNA was purified from those tumor samples and concentration adjusted after measurements performed with a spectrophotometer (Biophotometer, Eppendorf, Wesbury, N.Y., USA). Sequencing of the RNA samples was outsourced to BGI Tech Solutions (Tai Po, Hong Kong) who also performed data cleaning and quantitative analyses in a single blind manner.

CyTOF

Tumors harvested from in vivo experiments were processed into single cell suspensions and stored in freezing media (including 10% dimethyl sulfoxide) until they were stained for mass cytometry analyses.

Statistical Analyses

GraphPad Prism 8 (GraphPad Software, San Diego, Calif., USA) analysis tools were used to perform log rank Mantel-Cox test on Kaplan-Meier survival curves and Mann-Whitney test, as well as a mean to generate graphical representations of the data. SPSS Statistics 25 (IBM, Armonk, N.Y., USA) was the software used for analyses on tumor growth evolution based on daily measures of the tumor diameters as described before⁸. Statistical significance was claimed for p-values under 0.05.

Results

B16.OVA Tumor Model Respond to Anti-PD-1 but Fails does not Obtain Long-Term Responses

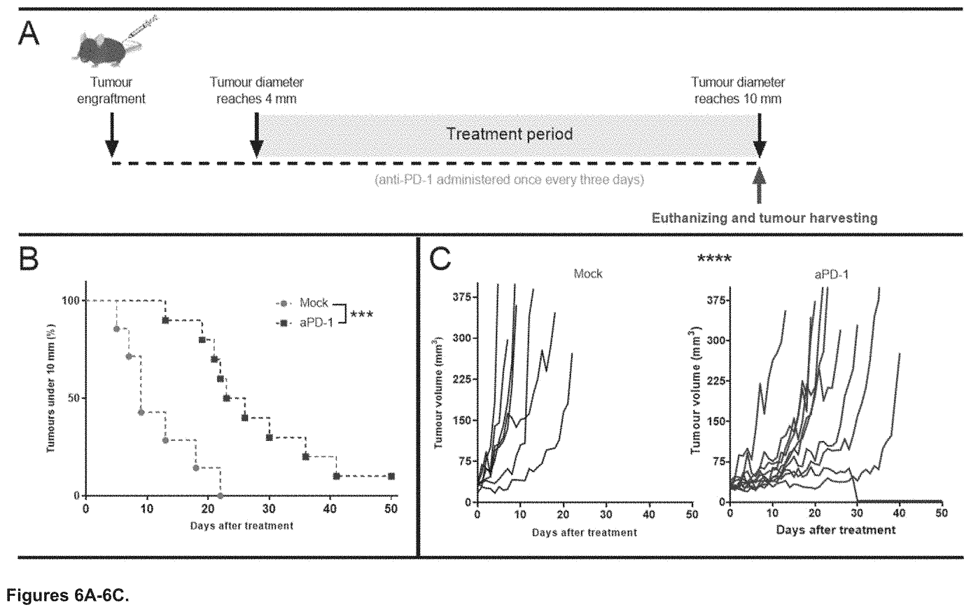

To study the mechanism of resistance to aPD-1, B16.OVA, a model that displays limited response to the inhibiting antibody⁸, was selected. In order to understand if there are intrinsic causes of resistance to the drug or if the tumor adapts to after the survival pressure provided by the drug, a tumor-growth criteria was adopted (FIG. 6). In that sense, the first time animals whose tumor reached at least 4 mm at maximum diameter were randomly assigned to either “Mock” or to “aPD-1” group. After that point, animals in the “aPD-1” group received PD-1 inhibiting antibody treatment systemically once every three days (at least 5 rounds in total). All the animals' tumors were measured daily until the moment when the maximum diameter was at least 10 mm. Animals with tumors surpassing that threshold were euthanized and tumors collected for further analysis.

While the size of the tumors at the start the treatment period and at the euthanizing were the same, the time for them to reach the second threshold was significantly different (p=0.0008) (FIG. 6B). The treatment had significant (p=0.0002) benefit in tumor growth control and one out of the ten animals treated with anti-PD-1 displayed an apparent complete response by day 30. Even if the treatment slowed tumor progression, 90% of the tumors eventually relapsed and reached the second threshold (FIG. 6C).

These results validate the presence of anti-tumor efficacy after PD-1 blockade but also the lack of long-term responses.

Tumors that Stop Responding to Anti-PD-1 Show Different Gene Expression Profiles than Tumors Naïve to the Therapy

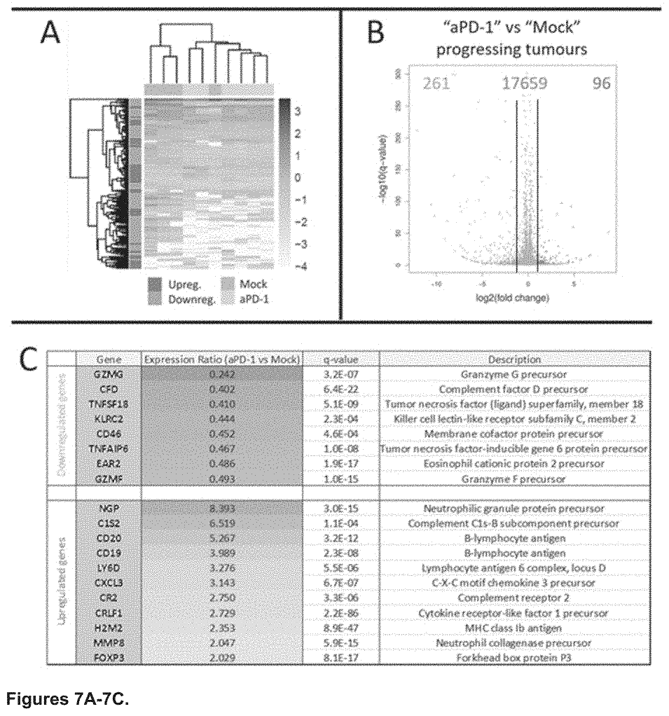

Tumors were collected as described in FIG. 6, subsequently processed and RNA extracted. Total RNA sequencing was performed and gene expression levels from each sample was quantified. For that analysis, four samples belonging to the “Mock” group and six samples from “aPD-1” group were randomly selected. After data cleaning, samples were arranged in a heatmap (FIG. 7A) and clustered based on the similarities between samples. This approach grouped samples from both groups apart with reasonable accuracy. The comparison of the expression profiles between groups exposed 357 genes differentially upregulated or downregulated (FIG. 7B). Out of those genes, 19 of them had marked immune nature (FIG. 7C).

From the downregulated immune-related genes with established function, 75% of them can be linked to T cells. Those genes include T-cell precursors (GZMG and GMZF), T-cell activity regulators (TNFSF18 [a.k.a. GITRL] and EAR2) and other T-cell proteins relevant in the interaction with other cell types (KLRC2) and immune components such as the complement (CD46). Besides T cells, other lymphocyte populations can be affected in a lesser extent by the downregulation such as NK cells or B cells.

Regarding the upregulated genes, the B-cell compartment is the population with higher number of genes (CD19, CD20, CR2, MMP8 and LY6D) followed by neutrophils (NGP, MMP8 and CXCL3). Complement-related genes are also noticeable in the upregulated genes (C1S2 and CR2). In contrast with the high number of T-cell related genes downregulated in aPD-1 refractory tumors, the only upregulated gene clearly associated with this cell population is FOXP3.

Among the whole list of differentially expressed genes in the aPD-1 refractory tumors, there is an observable trend indicating suppression of T-cell activities. Other cell populations are also affected but the results are not as clear.

The Use of a T-Cell Enabling Virotherapy Makes Anti-PD-1 Refractory Tumors Respond to Anti-PD-1

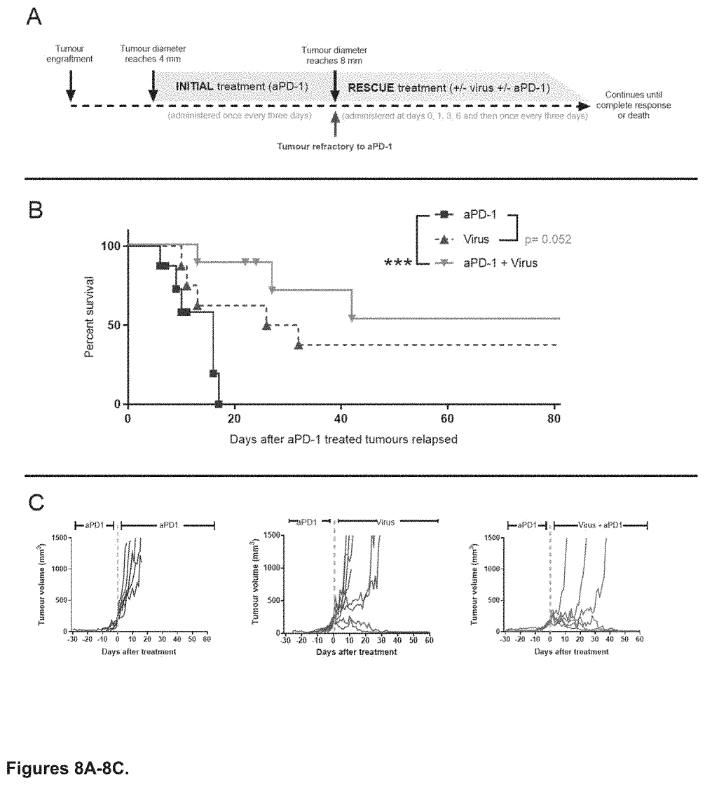

To study if tackling T-cell downregulation present in aPD-1 refractory tumors can result in better responses to the therapy, adenoviruses coding for two cytokines targeting anti-tumor T-cell activity (TNFalpha and IL-2). Similarly as described in FIG. 6A, animals carrying subcutaneous tumors were treated with aPD-1 (“initial treatment”) until they were considered refractory to the drug. After the refractory status is achieved, animals were randomised into “aPD-1” group where animals kept on receiving aPD-1, “Virus” group where animals received only virotherapy, or “aPD-1+ Virus” group where animals received aPD-1 and additionally virotherapy (“rescue treatment”). Treatments continued until maximum ethically allowed tumor volume (18 mm) or apparent complete responses (no visually noticeable lesions in the area) as described in FIG. 8A.

When studying tumor specific survival after those reached the refractory threshold (FIG. 8B), it was shown how T-cell enabling virotherapy can significantly (p=0.0009) increase survival and even trigger complete responses for 50% of the animals. When virotherapy alone was compared to aPD-1 as monotherapy, there are no significant improvement on survival, but some complete responses were achieved with the use of the above-mentioned viruses. Similar conclusions can be drawn from the analysis of individual tumor growth curves (FIG. 8C).

In addition, both survival and tumor growth data serve as a validation to the previously hypothesised aPD-1 refractory status of the tumors as in this experiment, the animals in “aPD-1” group kept on receiving the antibody after the refractory threshold is reached without any additional benefit.

aPD-1 Refractory Tumor Samples Treated with Virotherapy were Studied to Understand Immune Cell Impact of the Therapy

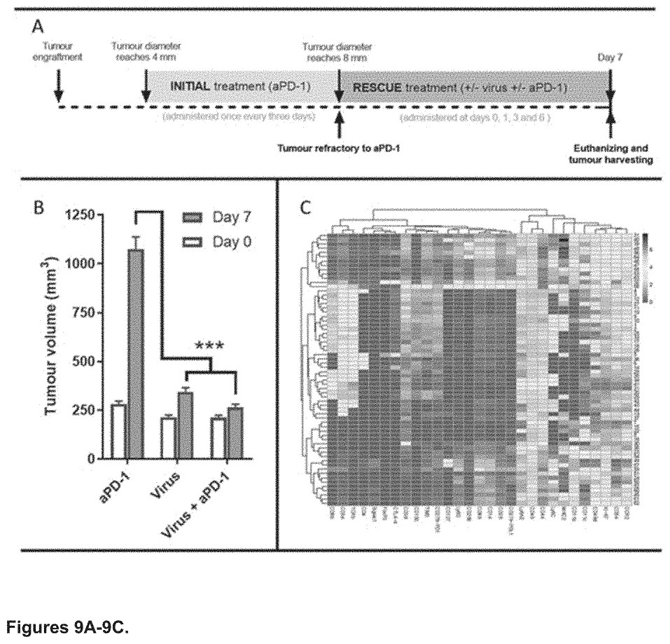

To generate samples subject to study for immune phenotyping of the tumors, a similar experimental design as in FIG. 8A was followed, although in this occasion, the “rescue treatment” given after refractory status is obtained will proceed for seven days (FIG. 9A). After that, tumors were collected and processed for analysis. Even if the tumors receiving the “rescue treatment” were similar, by day 7, there was a statistically significant (p<0.001) reduced tumor volume on when comparing animals from groups receiving virotherapy versus the aPD-1 treatment (FIG. 9B).

Those tumors collected at day 7, were used to investigate if the reappearing responses to aPD-1 were linked to a remodelling of the immune compartment present in the tumor microenvironment. In that sense, a mass cytometry analysis was run from the samples using 28 cell markers. Subsequently, 64 cell clusters were identified by using the FIowSOM algorithm on the CD45+ fraction of the cells. Those clusters were then represented in a heatmap (FIG. 9C) for further identification of the cell type and phenotype they represent.

Virotherapy Together with Anti-PD-1 Reshapes the Immune Microenvironment in Anti-PD-1 Refractory Tumors in Favor of Antitumor Responses

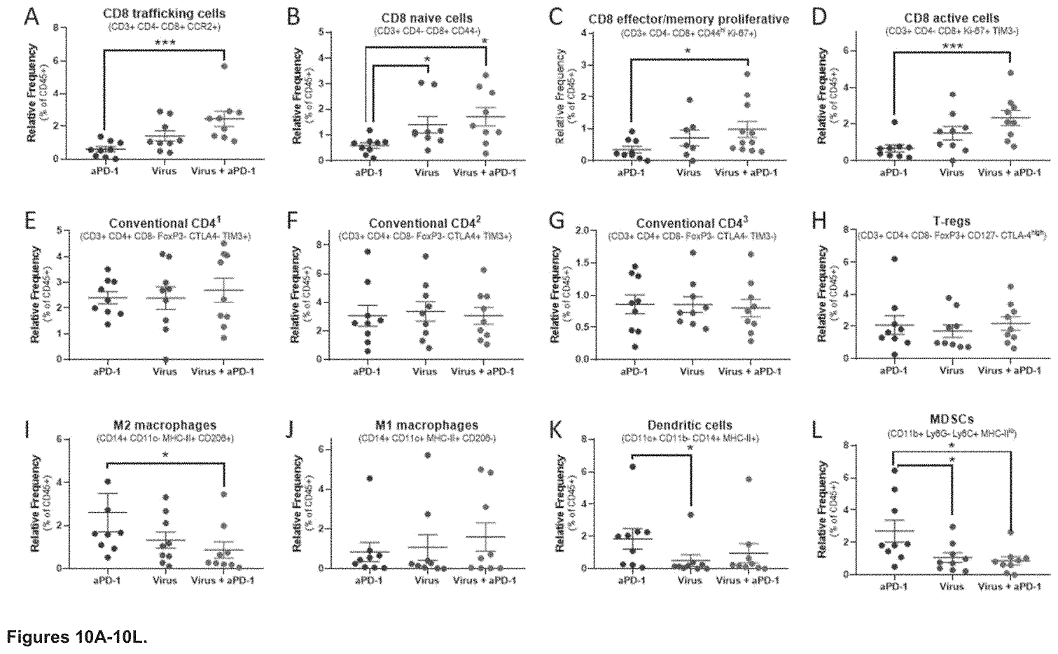

Cell clusters resulting from the mass cytometry were individually studied to determine the most likely cell type and phenotype. Those clusters with the clearest association to a specific cell population are shown in FIG. 10.

From all the 64 cell clusters, 29 of them had a T-cell phenotype based on CD45, CD3e and TCRb co-expression. Among those T-cell clusters, 22 were CD8+(CD4−) while 4 of them was CD4+(CD8-). Additionally, one cluster (number 4) was CD3e+TCRb+CD8+CD4+ and other two (clusters 42 and 43) were CD3e+TCRb+CD8−CD4−. Significant changes were only observed in CD8 T cell clusters but not in CD4, double positive or double negative T-cell clusters (FIG. 10A-H). Overall, 14 different CD8 T-cell subsets are significantly increased in tumors when they receive both aPD-1 and virotherapy. It can be observed how more T-cells with a phenotype linked to migration to inflamed sites (based on CCR2 marker) appear after the double therapy. Not only more trafficking of T cells but a wide T-cell presence is increased including not only effector/memory proliferative CD8 T cells (based on CD44 and Ki-67 markers), active and proliferating cells (based on Ki-67 and TIM-3 markers) but also naïve T-cells (based on CD44 marker). While the concomitant use of aPD-1 and virotherapy consistently displays an improved CD8 T cell distribution in the tumors, virotherapy alone does not provide the same degree of efficacy.

Regarding relevant myeloid populations, M2 macrophages and MDSCs are decreased in the tumors when virotherapy is added to the treatment. While no significant changes are observed in M1 macrophages or dendritic cells for the mentioned combination, dendritic cells are reduced when comparing virotherapy versus aPD-1 as monotherapies.

### Discussion

While none of the aPD-1 refractory tumors showed signs of response to aPD-1 as monotherapy, the inclusion of virotherapy with viruses coding for TNFalpha and IL-2 (in addition to aPD-1) triggered clear tumor growth control and even displayed complete responses. Those complete responses are remarkable as the challenge to reject those tumors not only appears from the refractory status but also because the tumor volume is around 8 times higher than the tumors initially treated with the checkpoint inhibitor. Higher initial tumor volumes encompass stronger immune and metabolic suppressive status. Interestingly, the results on survival together with the biological analyses on tumor samples indicate that, rather than exploiting a different mechanism of action to drive anti-tumor responses bypassing aPD-1 resistance, virotherapy is able to tackle the suppressive status of the tumor making them susceptible to PD-1 blockade.

### Example 3

In Vivo Experiments

To study the antitumor efficacy of virotherapy and checkpoint inhibition in a model of murine head and neck cancer, 1×10⁵ MOC2 murine oral cavity cancer cells were implanted subcutaneously on 4-6 week old female C57BL/6JOIaHsd mice (Envigo Labs). Twenty days after engraftment, animals were randomized into groups (n=7-8/group). Then they received systemic treatments of 0.1 mg of anti-PD-L1 (clone 10F.9G2, BE0101, BioXCell) every 3 days (starting from day 0) and intratumoral injections of 1×10⁸ viral particles (vp) (including equal amounts of Ad5-CMV-mIL2 and Ad5-CMV-mTNFa viruses, non-replicative in mice) on days 0, 1, 3, 6, 9, 12, 15 and 18. PBS was injected intratumorally for groups that did not receive virus. Tumor growth was followed until day 30. Survival of the animals was followed until day 90, where the anti-PD-L1 treatments continued once every three days, until maximum tumor size reached (18 mm) or complete tumor regression.

Cell Lines and Viruses

MOC2, a mouse oral squamous cell carcinoma cell line was cultured under recommended conditions. The cytokine-armed murine adenoviruses' (Ad5-CMV-mIL2 and Ad5-CMV-mTNFa) construction and production has been described previously (12) and were used in the in vivo experiments.

Statistical Analyses

GraphPad Prism 8 (GraphPad Software, San Diego, Calif., USA) analysis tools were used to perform log rank Mantel-Cox test on Kaplan-Meier survival curves and Mann-Whitney test, as well as a mean to generate graphical representations of the data. SPSS Statistics 25 (IBM, Armonk, N.Y., USA) was the software used for analyses on tumor growth evolution based on daily measures of the tumor diameters as described before(8). Statistical significance was claimed for p-values under 0.05.

Results

Virotherapy and Anti-PD-L1 Therapy Render the Best Antitumor Efficacy and Survival in a Model of Murine Head and Neck Cancer

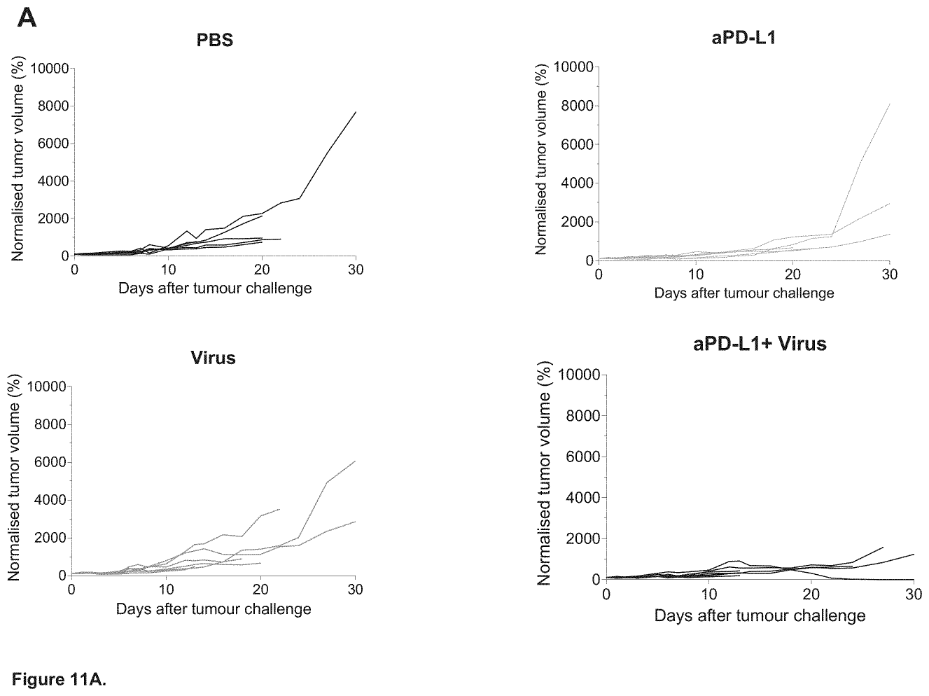

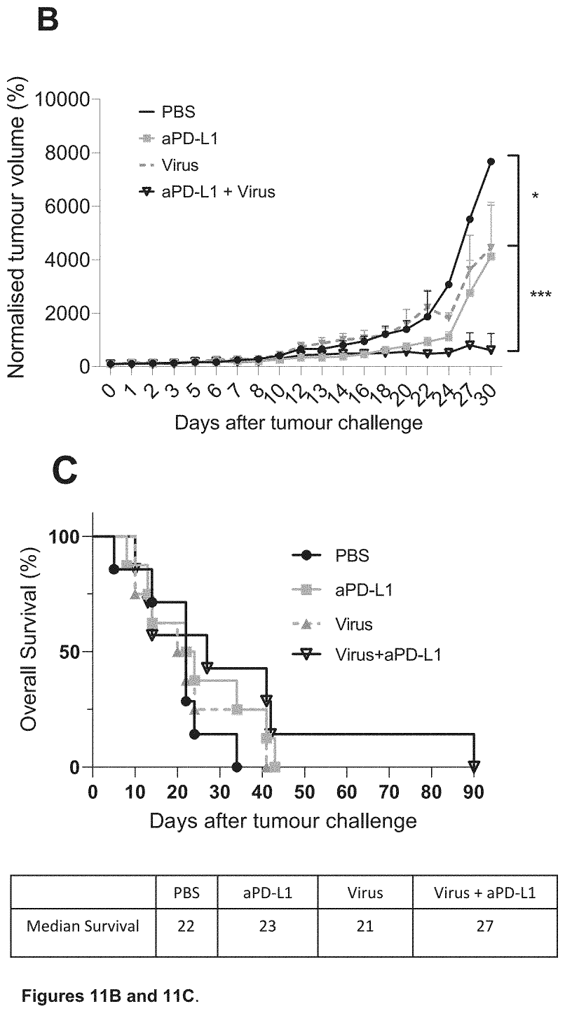

The therapeutic synergy of TNFa and IL-2-coding adenoviruses and anti-PD-L1 is not only limited to melanoma. In fact, we followed the antitumor effects of the combination in a model of murine oral cavity cancer during 30 days. As expected, individual tumor growth curves showed that intratumoral injection of PBS barely affected the tumor volumes (FIG. 11A). Administration of anti-PD-L1 (aPD-L1) or Ad5-CMV-mIL2+Ad5-CMV-mTNFa (virus) offered some additional therapeutic benefit in tumor bearing mice, with aPD-L1 interestingly providing a longer tumor volume control over time (FIG. 11A). Of note, the virus and aPD-L1 enabled fewer relapses and longer tumor growth control than any of the other tested therapies (FIG. 11A). This obviously enabled the combination therapy to provide an antitumor efficacy significantly better than the control or single-agent therapies (FIG. 11B). Importantly, mice receiving virus and anti-PD-L1 survived the longest and had better survival than PBS or single-agent treated mice (FIG. 11C).

### Discussion

Overall, these results show the synergy of the combination of TNFa and IL-2-coding adenoviruses and anti-PD-L1 inhibition in providing surprising and potent therapeutic activity in other in vivo tumor models, apart from melanoma.

### Example 4

Materials and Methods

Human Tumor Histocultures from Ovarian Cancer Samples

Ovarian cancer samples were collected from patients undergoing surgery, turned into single-cell suspension, following previously described methodology(9), and frozen up to −140° C. in freezing media containing 10% DMSO. After thawing, 3.5×10⁵ cells were seeded in 96-well plates and treated with 100 viral particles (vp) of Ad5/3-E2F-d24-hTNFa-IRES-hIL2 per cell, 20 μg/mL of anti-human PD-L1 (Avelumab, Evidentic), or the both in quadruplicates.

Cell Viability Assay

Human tumor histocultures were treated (as described before) up to 7 days. Cell viability was assessed on days 1, 5 and 7, with Cell Titer 96 AQueous One Solution Cell Proliferation Assay (Promega, G3582), following manufacturer indications. The viability of mock-treated cells was set to 100%.

Viruses

For the human tumor histoculture experiments, the oncolytic Ad5/3-E2F-d24-hTNFa-IRES-hIL2 (known as TILT-123) (11) was used.

Histopathology

After resection, patient-derived ovarian cancer tissue was processed for histopathology analysis. Tumor histologies were confirmed by a gynecological pathologist.

Statistical Analyses

GraphPad Prism 8 (GraphPad Software, San Diego, Calif., USA) analysis tools were used to perform unpaired t-test with Welch's correction, as well as a mean to generate graphical representations of the data. Statistical significance was claimed for p-values under 0.05.

Results

Combined Use of TILT-123 and Anti-PD-L1 Therapy Enables Fast and Potent Tumor Cell Killing in Patient-Derived Ovarian Cancer Tumor Histocultures

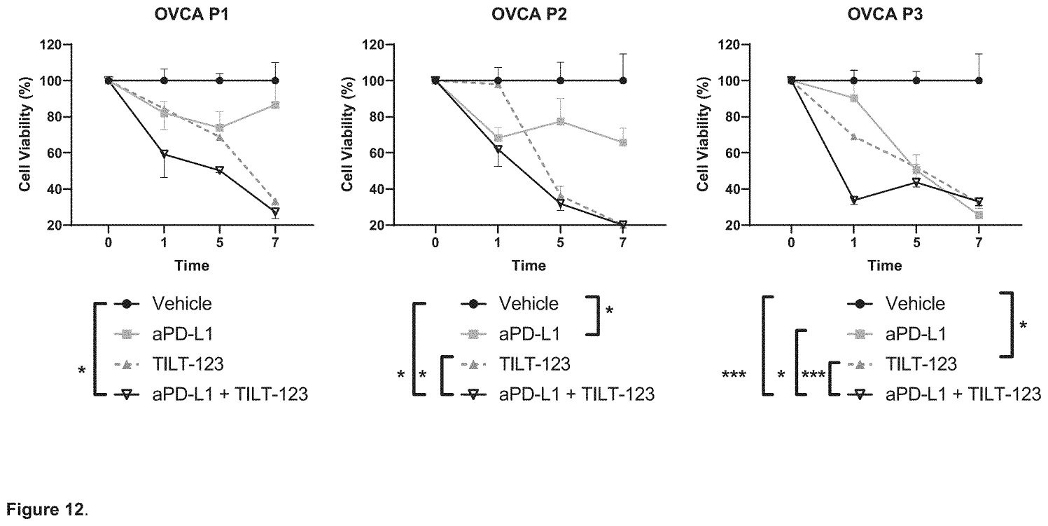

We also tested the combination treatment using Ad5/3-E2F-D24-TNFa-IRES-IL-2 (TILT-123) and anti-PD-L1 (aPD-L1), in tumor histocultures established from patient-derived ovarian cancer samples. These experiments allowed us to validate our combination strategy in a clinically-relevant tumor model, while taking into account the histological heterogeneity of human cancers. Upon treatment of the established ovarian cancer tumor histocultures (containing tumor and immune cells), we observed that the combination treatment enabled faster tumor-cell killing than the vehicle and/or single-agent treatments as evidenced in day 1 (FIG. 12). Of note, three distinct types of histologies were present in this experiment, being OVCA P1 ovarian low-grade serous carcinoma (stage IVB), OVCA P2 ovarian high-grade serous carcinoma (Stage IIIC) and OVCA P3 ovarian clear cell carcinoma (stage IVB).

### Discussion

Overall, these results show the surprising capabilities of TILT-123 to foster potent antitumor efficacy in combination with another anti-PD-L1 inhibitor (Avelumab) and also in another indication, such as ovarian cancer. Importantly, this therapeutic effect are independent of the histology of origin of the tissue.

### Example 5

Materials and Methods

Human Tumor Histocultures from a Brain Metastasis from a Squamous Cell Carcinoma of the Head and Neck Patient Refractory to Anti-PD-1 Therapy

A brain metastasis sample, from a squamous cell carcinoma of the head and neck patient refractory to anti-PD-1 therapy undergoing surgery, was collected, turned into single-cell suspension following previously described methodology(9). 3.5×10⁵ cells were freshly seeded in 96-well plates and treated with 100 viral particles (vp) per cell of Ad5/3-E2F-d24-hTNFa-IRES-hIL2, or Ad5/3-E2F-d24, or media (no virus), in triplicates.

Cell Viability Assay

Human tumor histocultures were treated (as described before) up to 7 days. Cell viability was assessed on day 3, 5 and 7, with Cell Titer 96 AQueous One Solution Cell Proliferation Assay (Promega, G3582), following manufacturer indications. The viability of mock-treated cells was set to 100%.

Viruses

For the human tumor histoculture experiments, the oncolytic Ad5/3-E2F-d24-hTNFa-IRES-hIL2 (known as TILT-123) or the oncolytic Ad5/3-E2F-D24 (11) was used.

Histopathology

Before undergoing resection, the patient has a confirmed base of tongue grade 3 primary tumor. Tumor histology were confirmed by a pathologist.

Statistical Analyses

GraphPad Prism 8 (GraphPad Software, San Diego, Calif., USA) analysis tools were used to perform unpaired t-test with Welch's correction, as well as a mean to generate graphical representations of the data. Statistical significance was claimed for p-values under 0.05.

Results

Single-Agent TILT-123 Induces Killing of Brain Metastasis Cells from a Patient Refractory to Anti-PD-1 Therapy

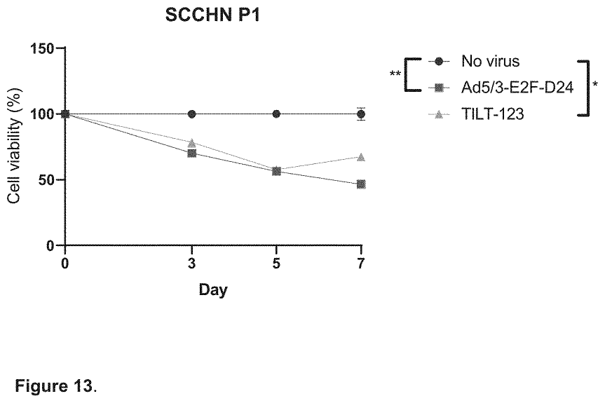

In a setting of squamous cell carcinoma of the head and neck refractory to anti-PD-1 therapy, the antitumor activity of TILT-123 is moderate (FIG. 13). Of note, TILT-123 was capable of significantly reducing the tumor cell content of the histoculture by day 7 compared to the no virus control (FIG. 13).

### Discussion

Overall, these data shows that the antitumor activity of single-agent TILT-123 is high but also limited in tumor cells from patients refractory to anti-PD-1 therapy. The latter highlights the need to combine TILT-123 with a checkpoint inhibitor to achieve improved therapeutic efficacy in such type of tumors.

### REFERENCES

1. Tumeh P C, Harview C L, Yearley J H, Shintaku I P, Taylor E J, Robert L, Chmielowski B, Spasic M, Henry G, Ciobanu V, West A N, Carmona M, et al. PD-1 blockade induces responses by inhibiting adaptive immune resistance. Nature 2014; 515: 568-71.
2. Nishino M, Ramaiya N H, Hatabu H, Hodi F S. Monitoring immune-checkpoint blockade: response evaluation and biomarker development. Nat Rev Clin Oncol 2017; 14: 655-68.
3. Ribas A, Robert C, Hodi F S, Wolchok J D, Joshua A M, Hwu W-J, Weber J S, Zarour H M, Kefford R, Loboda A, Albright A, Kang S P, et al. Association of response to programmed death receptor 1 (PD-1) blockade with pembrolizumab (MK-3475) with an interferon-inflammatory immune gene signature. J Clin Oncol 2015; 33: 3001-.
4. Herbst R S, Soria J C, Kowanetz M, Fine G D, Hamid O, Gordon M S, Sosman J A, McDermott D F, Powderly J D, Gettinger S N, Kohrt H E, Horn L, et al. Predictive correlates of response to the anti-PD-L1 antibody MPDL3280A in cancer patients. Nature 2014; 515: 563-567.
5. Fehrenbacher L, Spira A, Ballinger M, Kowanetz M, Vansteenkiste J, Mazieres J, Park K, Smith D, Artal-Cortes A, Lewanski C, Braiteh F, Waterkamp D, et al. Atezolizumab versus docetaxel for patients with previously treated non-small-cell lung cancer (POPLAR): a multicentre, open-label, phase 2 randomised controlled trial. Lancet 2016; 387: 1837-46.
6. Chen D S, Mellman I. Elements of cancer immunity and the cancer-immune set point. Nature 2017; 541: 321-30
7. White, E., Sabbatini, P., Debbas, M., Wold, W. S. M., Kusher, D. I., and Gooding, L. (1992). The 19-kilodalton adenovirus E1B transforming protein inhibits programmed cell death and prevents cytolysis by tumor necrosis factor alpha. Mol. Cell. Biol. 1992; 12: 2570-2580.
8. Cervera-Carrascon V, Siurala M, Santos J M, Havunen R, Tahtinen S, Karell P, et al. TNFa and IL-2 armed adenoviruses enable complete responses by anti-PD-1 checkpoint blockade. Oncoimmunology. 2018; 7(5):e1412902.
9. Taipale K, Tahtinen S, Havunen R, Koski A, Liikanen I, Pakarinen P, et al. Interleukin 8 activity influences the efficacy of adenoviral oncolytic immunotherapy in cancer patients. Oncotarget. 2018; 9(5):6320-35.
10. Santos J M, Havunen R, Siurala M, Cervera-Carrascon V, Tahtinen S, Sorsa S, et al. Adenoviral production of interleukin-2 at the tumor site removes the need for systemic postconditioning in adoptive cell therapy. International journal of cancer Journal international du cancer. 2017; 141(7):1458-68.
11. Havunen R, Siurala M, Sorsa S, Gronberg-Vaha-Koskela S, Behr M, Tahtinen S, et al. Oncolytic Adenoviruses Armed with Tumor Necrosis Factor Alpha and Interleukin-2 Enable Successful Adoptive Cell Therapy. Mol Ther Oncolytics. 2017; 4:77-86.
12. Siurala M, Havunen R, Saha D, Lumen D, Airaksinen A J, Tahtinen S, et al. Adenoviral Delivery of Tumor Necrosis Factor-alpha and Interleukin-2 Enables Successful Adoptive Cell Therapy of Immunosuppressive Melanoma. Molecular therapy: the journal of the American Society of Gene Therapy. 2016; 24(8):1435-43.

### CITED PATENT DOCUMENTS

WO2014170389

1. A method for treating cancer in a subject in need of such treatment comprising administering to the subject an effective amount of (a) an oncolytic adenoviral vector encoding TNFalpha and/or IL-2 as a transgene and (b) one or more immune checkpoint inhibitors.

2. The method of claim 1, wherein said one or more immune checkpoint inhibitors selectively binds to PD-L1 or PD-1.

3. The method of claim 1, wherein the oncolytic adenoviral vector is administered intratumorally, intravenously, intra-arterially, or intraperitoneally.

4. The method of claim 3, wherein the oncolytic adenoviral vector is administered intratumorally.

5. The method of claim 1, wherein the immune checkpoint inhibitor is a monoclonal antibody that selectively binds to PD-L1 selected from the group consisting of: BMS-936559, LY3300054, atezolizumab, durvalumab and avelumab.

6. The method of claim 1, wherein the immune checkpoint inhibitor is a monoclonal antibody that selectively binds to PD-1 selected from the group consisting of: pembrolizumab (MK-3475) and nivolumab (BMS-936558).

7. The method of claim 1, wherein the oncolytic adenoviral vector is administered in an amount from about 10⁶-10¹⁴ VP, 10⁶-10¹² VP, 10⁸-10¹⁴ VP, 10⁸-10¹² VP or 10¹⁰-10¹² VP.

8. The method of claim 1, wherein the one or more checkpoint inhibitors is administered in an amount from about 2 mg/kg to 25 mg/kg.

9. The method of claim 1, wherein the subject has a cancer selected from hepatocellular carcinoma, colorectal cancer, renal cell carcinoma, bladder cancer, lung cancer (including non-small cell lung cancer), stomach cancer, esophageal cancer, sarcoma, mesothelioma, melanoma, pancreatic cancer, head and neck cancer, ovarian cancer, breast cancer, cervical cancer and liver cancer.

10. The method of claim 9, wherein the subject has renal cell carcinoma or head and neck cancer.

11. The method of claim 1, wherein the subject has failed at least one previous chemotherapy or immunotherapy treatment.

12. The method of claim 11, wherein the subject has a cancer capable of mediating dysfunctionality of CD8+ T-cells.

13. The method of claim 11, wherein the subject has a cancer having at least one gene related to immune activity selected from the group consisting of GZMG, GMZF, KLRC2 and CD46 and a T-cell activity regulator selected from the group consisting of TNFSF18/GITRL and EAR2 that is downregulated.

14. The method of claim 11, wherein the subject has an anti-PD-1 refractory cancer.

15. The method of claim 1, comprising administering to the subject an additional therapy selected from the group consisting of radiotherapy, chemotherapy, antiangiogenic agents or targeted therapies, such as alkylating agents, nucleoside analogs, cytoskeleton modifiers, cytostatic agents, monoclonal antibodies, and kinase inhibitors.

16. The method of claim 1, wherein the subject is a human.

17. The method of claim 1, wherein a first dose of the oncolytic adenoviral vector and a first dose of the immune checkpoint inhibitor are simultaneously administered to the subject.

18. The method of claim 1, wherein a first dose of the oncolytic adenoviral vector and a first dose of the immune checkpoint inhibitor are administered sequentially in 24 hours.

19. The method of claim 1, wherein said oncolytic adenoviral vector encoding TNFalpha and/or IL-2 as a transgene comprises
a 5/3 chimeric fiber knob,
E2F1 promoter for tumor specific expression of E1A,
a 24 bp deletion (D24) in the Rb binding constant region 2 of adenoviral E1,
a nucleic acid sequence deletion of viral gp19k and 6.7k reading frames, and
a nucleic acid sequence encoding at least TNFalpha and/or IL-2 as transgene(s) in the place of the deleted gp19k/6.7K in the E3 region resulting in replication-associated control of transgene expression under the viral E3 promoter.

20. The method of claim 19, wherein said oncolytic adenoviral vector encoding TNFalpha and/or IL-2 as a transgene further comprises a deletion of E1B19k.

21. A pharmaceutical composition comprising (a) an oncolytic adenoviral vector encoding TNFalpha and/or IL-2 as a transgene and (b) one or more immune checkpoint inhibitors.

22. The pharmaceutical composition according to claim 21, wherein said one or more immune checkpoint inhibitors selectively binds to PD-L1 or PD-1.

23. The pharmaceutical composition of claim 21, wherein said oncolytic adenoviral vector encoding TNFalpha and/or IL-2 as a transgene comprises
a 5/3 chimeric fiber knob,
E2F1 promoter for tumor specific expression of E1A,
a 24 bp deletion (D24) in the Rb binding constant region 2 of adenoviral E1,
a nucleic acid sequence deletion of viral gp19k and 6.7k reading frames, and
a nucleic acid sequence encoding at least TNFalpha and/or IL-2 as transgene(s) in the place of the deleted 19k/6.7K in the E3 region resulting in replication-associated control of transgene expression under the viral E3 promoter.

24. A kit which comprises a first container, a second container and a package insert, wherein the first container comprises at least one dose of a pharmaceutical composition containing an oncolytic adenoviral vector encoding TNFalpha and/or IL-2 as a transgene, the second container comprises at least one dose of a pharmaceutical composition comprising a checkpoint inhibitor, and the package insert comprises instructions for treating an individual having cancer using the pharmaceutical composition(s).

25. The kit according to claim 24, wherein said oncolytic adenoviral vector encoding TNFalpha and/or IL-2 as a transgene comprises
a 5/3 chimeric fiber knob,
E2F1 promoter for tumor specific expression of E1A,
a 24 bp deletion (D24) in the Rb binding constant region 2 of adenoviral E1,
a nucleic acid sequence deletion of viral gp19k and 6.7k reading frames, and
a nucleic acid sequence encoding at least TNFalpha and/or IL-2 as transgene(s) in the place of the deleted gp19k/6.7K in the E3 region resulting in replication-associated control of transgene expression under the viral E3 promoter.

26. The kit of claim 24, wherein said oncolytic adenoviral vector encoding TNFalpha and/or IL-2 as a transgene further comprises a deletion of E1B19k.

27-46. (canceled)

47. The composition of claim 21, wherein said oncolytic adenoviral vector encoding TNFalpha and/or IL-2 as a transgene further comprises a deletion of E1B19k.


SEQUENCE LISTING (5 sequences)

SEQ ID NO: 1 - DNA, 531 bp, adenovirus
atggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatga

SEQ ID NO: 2 - DNA, 390 bp, adenovirus
atggaggcttgggagtgtttggaagatttttctgctgtgcgtaacttgctggaacagctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatga

SEQ ID NO: 3 - DNA, 680 bp, adenovirus
tggtaccatccggacaaagcctgcgcgcgccccgccccgccattggccgtaccgccccgcgccgccgccccatcccgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcgccctcgagctagaagcttgttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggag

SEQ ID NO: 4 - DNA, 27458 bp, adenovirus
gtgatcgatccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggatggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacg

SEQ ID NO: 5 - DNA, 3712 bp, adenovirus
tggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactgaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg